SEQUENCE LISTING (10 sequences)

SEQ ID NO: 1 - DNA, 18 bp, Artificial Sequence
  synthetic sequence
tcaggacccacaggagcg

SEQ ID NO: 2 - DNA, 22 bp, Artificial Sequence
  synthetic sequence
cctcacgtcgcagtaactgttg

SEQ ID NO: 3 - DNA, 32 bp, Artificial Sequence
  synthetic sequence
cccagaaagttaccacagttatgcacagagct

SEQ ID NO: 4 - DNA, 20 bp, Artificial Sequence
  synthetic sequence
ccggacagagcccattacaa

SEQ ID NO: 5 - DNA, 23 bp, Artificial Sequence
  synthetic sequence
cgaatgtctacgtgtgtgctttg

SEQ ID NO: 6 - DNA, 27 bp, Artificial Sequence
  synthetic sequence
cgcacaaccgaagcgtagagtcacact

SEQ ID NO: 7 - DNA, 25 bp, Artificial Sequence
  synthetic sequence
tcacccacactgtgcccatctacga

SEQ ID NO: 8 - DNA, 25 bp, Artificial Sequence
  synthetic sequence
cagcggaaccgctcattgccaatgg

SEQ ID NO: 9 - DNA, 26 bp, Artificial Sequence
  synthetic sequence
atgccctcccccatgccatcctgcgt

SEQ ID NO: 10 - DNA, 18 bp, Artificial Sequence
  synthetic sequence
ccgtcaattcmtttragt

(19) United States
(12) Patent Application Publication
(10) Pub. No.: US 2018/0223338 A1
(43) Pub. Date: Aug. 9, 2018

(54) MICROBIOME IDENTIFICATION AND USE IN DIAGNOSIS OF HEAD AND NECK SQUAMOUS CELL CANCER

(71) Applicant: THE JOHNS HOPKINS UNIVERSITY, Baltimore, MD (US)

(72) Inventors: Rafael Guerrero-Preston, Baltimore, MD (US); David Sidransky, Baltimore, MD (US)

(21) Appl. No.: 15/889,800

(22) Filed: Feb. 6, 2018

Related U.S. Application Data
(60) Provisional application No. 62/455,848, filed on Feb. 7, 2017.

(51) Int. Cl. C12Q 1/689; C12Q 1/6886; G16H 50/50; G16H 50/20

(52) U.S. Cl. CPC C12Q 1/689; C12Q 1/6886; G16H 50/50; C12Q 2600/118; C12Q 2600/16; C12Q 2600/166; G16H 50/20

(57) ABSTRACT

Methods for analyzing the bacterial microbiome of a subject and triaging the subject into treatment for head and neck squamous cell cancer from a saliva or tissue sample are provided. The methods also provide means for differentiation of different cancer patient cohorts from the bacterial microbiome of a subject.

### REFERENCE TO RELATED APPLICATIONS

This application claims the benefit of U.S. Provisional Patent Application No. 62/455,848, filed on Feb. 7, 2017, which is hereby incorporated by reference for all purposes as if fully set forth herein.

### STATEMENT OF GOVERNMENTAL INTEREST

This invention was made with government support under grant nos. CA164092, CA084986, CA121113, DE019032, and DE020957, awarded by the National Institutes of Health. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Feb. 7, 2017, is named P14581-01_ST25.txt and is 2,154 bytes in size.

### BACKGROUND OF THE INVENTION

Microorganisms cause an estimated 20% of human cancer. The best-known example is the role Helicobater pylori plays in gastric cancer. DNA and RNA viruses have also been identified as etiological factors in cervical, oropharyngeal, nasopharyngeal, and hepatocellular carcinoma. A handful of laboratories have reported links between the human microbiome, immuno modulators, inflammation and tumor initiation or progression in oral, colon, pancreatic, liver, esophageal and prostate cancers. The biological mechanism of these associations is not yet understood.

Cancer initiation, development, metastasis and its response to therapy are shaped by site specific genomic, epigenomic and immunologic alterations, all of which are related to acute or chronic inflammatory states. Four different types of inflammation seem to precede cancer initiation: 1) Chronic inflammation associated to infections or autoimmune disorders; 2) Low grade chronic inflammation associated to environmental irritants, health behaviors, or obesity; 3) Tumor associated inflammation; 4) Therapy-induced inflammation. Cancer initiation and progression by oncogenic mutations, genomic instability, early tumor promotion, and enhanced angiogenesis are changes linked to chronic inflammation associated to multiple etiologies. Tumor-associated inflammation enhances angiogenesis, promotes tumor progression and metastatic spread, and causes local immunosuppression. Therapy-induced inflammation is linked to trauma, necrosis, and tissue injury, which stimulate tumor re-emergence and resistance to therapy. Conversely, therapy-induced inflammation can also enhance antigen presentation, leading to immune-mediated tumor eradication.

Most pathogen-induced tumors are preceded by pathogen, tissue and site specific host-mediated inflammatory states, which lead to pre-malignant dysplastic, metaplastic and/or fibrotic changes, first described by Virchow 150 years ago. The normal tissue-repair response to injury and infection is orchestrated by an evolutionary conserved sequence of molecular changes triggered by pattern-recognition receptors (PRRs), many of which belong to the Toll-like receptor (TLR) famil. PPRs recognize pathogen associated molecular patterns (PAMPs) or damage associated molecular patterns (DAMPs) setting off a cascade of events that activate the innate immune response. There are several mechanisms by which bacterial infection can lead to the initiation and progression of oncogenic processes. Bacterial endotoxins, metabolic byproducts of bacterial infection, and increased enzymatic activity as a result of bacterial infection, can induce somatic mutations and signaling pathway alterations. The role of microbes and viruses in cancer development is also associated to a wide spectrum of focalized changes driven by innate and adaptive immune responses.

High-throughput technologies are increasingly being used to test hypotheses and build experimental strategies aimed at revealing the role of bacteria in health and disease. The method of excellence for performing initial microbiome characterization, which bypasses the need for culturing, is the use of small-subunit ribosomal RNA (rRNA) studies, in which the 16S rRNA gene sequences (for archaea and bacteria) are used as stable phylogenetic markers to identify taxonomic lineages in a given sample. The 16S rRNA gene has nine variable regions, including a combination of variable and moderately conserved regions optimal for performing analyses at different phylogenetic depths. The V3-V5 region of the 16S rRNA gene is one of the preferred regions for characterizing the communities with few errors for taxonomy assignment.

Head neck squamous cell carcinomas (HNSCC) is a diverse group of tumor types, originally classified by anatomic subsite, but more recently best understood in terms of etiology, molecular drivers and immune phenotype. During the past twenty years the genomic and epigenomic changes in tumor development and treatment of HNSCC have been mapped. These studies have revealed that the inactivation of the p53 and retinoblastoma (pRb) pathways is one of the earliest change seen in both, Human Papilloma Virus (HPV) negative HNSSC patients, who have accumulated hundreds of somatic mutations and promoter methylation of tumor suppressor genes (TSG), and in HPV positive HNSCC, which have a low frequency of somatic mutations, a different pattern of promoter methylation in TSG, and express the HVP oncogenes E6 and E7.

Recently, the present inventors used 16S rRNA-sequencing to unveil novel characteristics of the saliva microbiome from head and neck cancer patients treated for Human Papilloma Virus (HPV) positive oropharyngeal cancer, and HPV negative oropharyngeal and oral cavity cancer (WO2015/153566) and incorporated by reference herein. Longitudinal analyses of samples taken before and after surgery, revealed a reduction in the alpha diversity measure after surgery, together with an increase of this measure in patients that recurred (p<0.05). HNSCC patients had a significant loss in richness and diversity of microbiota species (p<0.05) compared to the controls. Overall, the Operational Taxonomic Units (OTU) network shows that the relative abundance of OTU's within genus Streptococcus, Dialister, and Veillonella can be used to discriminate HNSCC from control samples (p<0.05). Tumor samples lost Neisseria, Aggregatibacter (Proteobacteria), Haemophillus (Firmicutes) and Leptotrichia (Fusobacteria). Paired taxa within family Enterobacteriaceae, together with genus Oribacterium, distinguish OCSCC samples from OPSCC and normal samples (p<0.05). Similarly, only HPV positive samples have an abundance of genus Gemellaceae and Leuconostoc (p<0.05).

However, there still exists a need to provide non-invasive methods for identifying patients at risk for HNSCC or other oral and squamous cell cancers.

### SUMMARY OF THE INVENTION

The present inventors performed novel analyses of the 16S rRNA-sequencing data that allowed identification of associations between the saliva microbiome and tumor characteristics in the same HNSCC patient population. The present inventive methods involved use the Resphera Insight pipeline to perform a cross-sectional comparison at the species level of the microbial communities present in saliva DNA from HPV positive and HPV negative patients with cancer of the oropharynx, cancer of the oral cavity, and participants with normal oral cavity epithelium. Between about 1-4 additional saliva samples were collected in subsequent visits, from 10 of the 19 HNSCC patients to evaluate the longitudinal association between microbial species abundance and community members and treatment effects. The results were then validated on 59 samples from our Discovery cohort on 514 samples from the Human Microbiome Project (HMP).

As such, in accordance with an embodiment, the present invention provides methods for identifying the bacterial taxonomic profile in a subject suspected of having HNSCC or having HNSCC, comprising: a) obtaining a biological sample from the subject; b) isolating the nucleic acid in the sample; c) amplifying the 16S rRNA V3-V5 gene region of bacterial nucleic acid present in the sample of b) using specific primers and probes for the 16S rRNA V3-V5 region; d) sequencing the amplified DNA of step c); and e) identifying the bacterial species present in the sample using high-resolution profiling of the sequences of step d).

In accordance with another embodiment, the present invention provides methods for diagnosis and treatment of patients with HNSCC, neck cancer of the oropharynx, cancer of the oral cavity comprising the steps of: (a) obtaining nucleic acid from a saliva or tissue sample taken from the subject; (b) amplifying the 16S rRNA V3-V5 gene region of bacterial nucleic acid present in the nucleic acid of step (a); (c) sequencing the amplified DNA of step (b); using high-resolution profiling of the sequences of step (c) and a comparison to bacterial species present in a reference or control sample that correlates to normal mucosa or saliva from patients with normal mucosa; (e) comparing the bacterial species present in saliva or tissue, as well as its microbial diversity, richness and/or abundance and identifying subjects with HNSCC from subjects without HNSCC; or, HNSCC patients with oral squamous cell carcinoma from patients with oropharyngeal carcinoma; or high-risk HPV positive oropharyngeal carcinoma patients from high-risk HPV negative oropharyngeal carcinoma patients. or, HNSCC patients who have been treated with surgery from HNSCC patients who have been treated with surgery and chemoradiation or with PD-1 checkpoint blockade therapy; or, tumor recurrence in HNSCC patients treated with surgery, chemoradiation, surgery and chemoradiation.

As such, in accordance with another embodiment, the present invention provides methods for diagnosis and treatment of patients with HNSCC, comprising: a) obtaining a biological sample from the subject; b) isolating the nucleic acid in the sample; c) amplifying the 16S rRNA V3-V5 gene region of bacterial nucleic acid present in the sample of b) using specific primers and probes for the 16S rRNA V3-V5 region; d) sequencing the amplified DNA of step c); e) identifying the bacterial species present in the sample using high-resolution profiling of the sequences of step d); f) identifying the subject as having HNSCC when one or more bacterial species selected from the group consisting of Lactobacillus gasseri, Lactobacillus johnsonii, Haemophilus parainfluenza, Lactobacillus fermentum, L. rhamnosus and Fusobacterium periodonticum is present in the sample.

In accordance with a further embodiment, the present invention provides method of identifying a subject having HNSCC as having oropharyngeal squamous cell cancer (OPSCC) comprising the steps of: a) obtaining a biological sample from the subject; b) isolating the nucleic acid in the sample; c) amplifying the 16S rRNA V3-V5 gene region of bacterial nucleic acid present in the sample of b) using specific primers and probes for the 16S rRNA V3-V5 region; d) sequencing the amplified DNA of step c); e) identifying the bacterial species present in the sample using high-resolution profiling of the sequences of step d); f) identifying the subject as having OPSCC when one or more bacterial species selected from the group consisting of Lactobacillus gasseri, and Lactobacillus johnsonii.

### BRIEF DESCRIPTION OF THE DRAWINGS

FIG. 1 is a table showing the characteristics of head and neck cancer patients used in the study.

FIG. 2 is a table showing patient characteristics for repeated samples analysis.

FIGS. 3A-3C: taxonomic profiles at the phyla (3A) and species (3B) levels for both control and HNSCC samples. 3C is a hierarchical clustering of taxonomic profiles (top 50 species level calls).

FIGS. 4A-4B: Beta diversity through Principal Component Analysis (PCA) with Bray-Curtis distances, comparing HNSCC (n=17), Control (n=25) and HMP samples (n=541, saliva=292, mid vagina=128 and introitus n=121). Panel 4A. Comparison between controls and HNSCC patients. Panel 4B. Comparison of Control, tumors and HMP samples.

FIGS. 5A-5C: Taxonomic profiles at the phyla (5A) and species (5B) levels for controls, HNSCC samples and HMP saliva, mid vagina and introitus. (5C) depicts bar graphs showing the percentage abundance of the different lactobacillus species in the different groups.

FIGS. 5D-5I: Bar graphs that depict reads of Lactobacillus gasseri, and Lactobacillus johnsonii in saliva samples from HNSCC patients including oropharyngeal and oral cancer patients (5D), saliva samples from HPV positive and HPV negative patients (5E), saliva samples from patients with Stage Iva or higher HNSCC (5F), HNSCC patients before and after surgery (5G), HNSCC patients treated with surgery and chemoradiation (5H), and male HNSCC patients with surgery alone (5I).

FIGS. 6A-6F: represents species that differed significantly in relative abundance (p<0.05) when comparing saliva from normal patients with patients with HNSCC, normal saliva and vaginal flora samples from HMP Rarefaction curves of species richness between controls, HNSCC and HMP samples based on chaol richness estimator. Pie charts were created showcasing taxa distinguishing samples by Histology and recruitment site. The OTU network (6A) and pie charts shows the total abundance of species in saliva from HNSCC patients (RGP HNSCC—6B), saliva from JHU normal participants (RGP Control—6C), HMP saliva (FIG. 6D), as well as microbiome samples from mid vagina (6E) and the vaginal introitus (6F).

FIGS. 7A-7D: The relative enrichment of Fusobacterium nucleatum in HNSCC saliva when compared to controls from Hopkins and HMP (7A) is only seen in patients with advanced disease (T3>) (7B) and only in patients treated with surgery and chemo-radio-therapy (7C) when compared with patients only treated with surgical removal of the tumor (7D).

FIGS. 8A-8C depict bar graphs of the reads in HNSCC patient saliva samples showing the relative enrichment of Fusobacterium nucleatum in HNSCC saliva when compared to controls from Hopkins and HMP (8A), patients with advanced disease (T3>) (8B), and patients treated with surgery and chemo-radio-therapy when compared with patients only treated with surgical removal of the tumor (8C).

### DETAILED DESCRIPTION OF THE INVENTION

Tests can be carried out on any suitable sample that is likely to yield squamous cells or squamous cell nucleic acids. Particular samples which can be used include tissue specimens, biopsy specimens, surgical specimens, saliva, nasal mucosa, leukoplakia, erythroplakia, leukoerythroplakia and cytological specimens. It may be beneficial to extract nucleic acids from the cells prior to testing. Some techniques of testing may not require pre-extraction. Some testing may be done on proteins which may or may not be extracted from the cells prior to testing for particular detection techniques.

As such, in accordance with another embodiment, the present invention provides methods for identifying the bacterial taxonomic profile in a subject suspected of having HNSCC or having HNSCC, comprising: a) obtaining a biological sample from the subject; b) isolating the nucleic acid in the sample; c) amplifying the 16S rRNA V3-V5 gene region of bacterial nucleic acid present in the sample of b) using specific primers and probes for the 16S rRNA V3-V5 region; d) sequencing the amplified DNA of step c); and e) identifying the bacterial species present in the sample using high-resolution profiling of the sequences of step d).

In accordance with an embodiment, the present invention provides methods for triaging a patient suspected of having HNSCC into treatment, comprising: a) obtaining a biological sample from the subject; b) isolating the nucleic acid in the sample; c) amplifying the 16S rRNA V3-V5 gene region of bacterial nucleic acid present in the sample of b) using specific primers and probes for the 16S rRNA V3-V5 region; d) sequencing the amplified DNA of step c); e) identifying the bacterial species present in the sample using high-resolution profiling of the sequences of step d); and 0 triaging the subject suspected of having HNSCC into treatment for HNSCC when one or more bacterial species selected from the group consisting of Lactobacillus gasseri, Lactobacillus johnsonii, Haemophilus parainfluenza, Lactobacillus fermentum, L. rhamnosus and Fusobacterium periodonticum is present in the sample.

In accordance with a further embodiment, the present invention provides method of triaging a subject having HNSCC into treatment for oropharyngeal squamous cell cancer (OPSCC) comprising the steps of: a) obtaining a biological sample from the subject; b) isolating the nucleic acid in the sample; c) amplifying the 16S rRNA V3-V5 gene region of bacterial nucleic acid present in the sample of b) using specific primers and probes for the 16S rRNA V3-V5 region; d) sequencing the amplified DNA of step c); e) identifying the bacterial species present in the sample using high-resolution profiling of the sequences of step d); f) triaging the subject as having OPSCC into treatment when one or more bacterial species selected from the group consisting of Lactobacillus gasseri, and Lactobacillus johnsonii.

In accordance with another embodiment of the present invention, it will be understood that the term “biological sample” or “biological fluid” includes, but is not limited to, any quantity of a substance from a living or formerly living patient or mammal. Such substances include, but are not limited to, blood, serum, plasma, urine, cells, organs, tissues, bone, bone marrow, lymph, lymph nodes, synovial tissue, chondrocytes, synovial macrophages, endothelial cells, and skin.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

The nucleic acids used as primers in embodiments of the present invention can be constructed based on chemical synthesis and/or enzymatic ligation reactions using procedures known in the art. See, for example, Sambrook et al. (eds.), Molecular Cloning, A Laboratory Manual, 3^{rd} Edition, Cold Spring Harbor Laboratory Press, New York (2001) and Ausubel et al., Current Protocols in Molecular Biology, Greene Publishing Associates and John Wiley & Sons, NY (1994). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleotide sequences used herein are those which hybridize under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C.

The term “isolated and purified” as used herein means a protein that is essentially free of association with other proteins or polypeptides, e.g., as a naturally occurring protein that has been separated from cellular and other contaminants by the use of antibodies or other methods or as a purification product of a recombinant host cell culture.

The term “biologically active” as used herein means an enzyme or protein having structural, regulatory, or biochemical functions of a naturally occurring molecule.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In accordance with one or more embodiments of the present invention, it will be understood that the types of cancer diagnosis which may be made, using the methods provided herein, is not necessarily limited. For purposes herein, the cancer can be any cancer. As used herein, the term “cancer” is meant any malignant growth or tumor caused by abnormal and uncontrolled cell division that may spread to other parts of the body through the lymphatic system or the blood stream.

The cancer can be a metastatic cancer or a non-metastatic (e.g., localized) cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of diagnosis, staging, screening, or other patient management, including treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules.

“Differential expression” may mean qualitative or quantitative differences in the temporal and/or cellular gene expression patterns within and among cells and tissue. Thus, a differentially expressed gene may qualitatively have its expression altered, including an activation or inactivation, in, e.g., normal versus disease tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in both. Alternatively, the difference in expression may be quantitative, e.g., in that expression is modulated, either up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, northern analysis, and RNase protection.

“Identical” or “identity” as used herein in the context of two or more nucleic acids or polypeptide sequences may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

“Substantially complementary” used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

“Target” as used herein can mean an oligonucleotide or portions or fragments thereof, which may be bound by one or more probes under stringent hybridization conditions. “Target” as used herein may also mean a specific 16S rRNA V3-V5 region of a bacterial genome, or portions or fragments thereof, which may be bound by one or more probes under stringent hybridization conditions.

A probe is also provided comprising a nucleic acid described herein. Probes may be used for screening and diagnostic methods, as outlined below. The probes may be attached or immobilized to a solid substrate or apparatus, such as a biochip.

The probe may have a length of from 8 to 500, 10 to 100 or 20 to 60 nucleotides. The probe may also have a length of at least 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 nucleotides. The probe may further comprise a linker sequence of from 10-60 nucleotides.

A biochip is also provided. The biochip is an apparatus which, in certain embodiments, comprises a solid substrate comprising an attached probe or plurality of probes described herein. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. In an embodiment, two or more probes per target sequence are used. The probes may be capable of hybridizing to target sequences associated with a single disorder.

The probes may be attached to the biochip in a wide variety of ways, as will be appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linkers. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

A method of identifying a nucleic acid associated with one or more species of bacteria that are associated with a disease or a pathological condition is also provided. The method comprises measuring a level of the nucleic acid in a sample that is different than the level of a control. In accordance with an embodiment, the nucleic acid is a 16S rRNA from the V3-V5 region of a bacterial genome, and the detection may be performed by contacting the sample with a probe or biochip described herein and detecting the amount of hybridization. PCR may be used to amplify nucleic acids in the sample, which may provide higher sensitivity.

The target nucleic acid or portions or fragments thereof, associated with one or more species of bacteria that are associated with a disease or a pathological condition may also be detected by immobilizing the nucleic acid to be examined on a solid support such as nylon membranes and hybridizing a labeled probe with the sample. Similarly, the target nucleic or portions or fragments thereof, may also be detected by immobilizing the labeled probe to a solid support and hybridizing a sample comprising a labeled target nucleic acid. Following washing to remove the non-specific hybridization, the label may be detected.

The target nucleic acid or portions or fragments thereof, associated with one or more species of bacteria that are associated with a disease or a pathological condition may also be detected in situ by contacting permeabilized cells or tissue samples with a labeled probe to allow hybridization with the target nucleic acid. Following washing to remove the non-specifically bound probe, the label may be detected.

The detection of the target nucleic acid, or portions or fragments thereof, associated with one or more species of bacteria that are associated with a disease or a pathological condition can be through direct hybridization assays or can comprise sandwich assays, which include the use of multiple probes, as is generally known in the art.

A variety of hybridization conditions may be used, including high, moderate and low stringency conditions as outlined above. The assays may be performed under stringency conditions which allow hybridization of the probe only to the target. Stringency can be controlled by altering a step parameter that is a thermodynamic variable, including, but not limited to, temperature, formamide concentration, salt concentration, chaotropic salt concentration pH, or organic solvent concentration.

Hybridization reactions may be accomplished in a variety of ways. Components of the reaction may be added simultaneously, or sequentially, in different orders. In addition, the reaction may include a variety of other reagents. These include salts, buffers, neutral proteins, e.g., albumin, detergents, etc. which may be used to facilitate optimal hybridization and detection, and/or reduce non-specific or background interactions. Reagents that otherwise improve the efficiency of the assay, such as protease inhibitors, nuclease inhibitors and anti-microbial agents may also be used as appropriate, depending on the sample preparation methods and purity of the target.

A kit is also provided comprising an array of oligonucleotides as described herein, or portions or fragments thereof, for 16S rRNA V3-V5 regions associated with one or more species of bacteria that are associated with a disease or a pathological condition as well as a biochip as described herein. They are typically in a package which contains all elements, optionally including instructions. Instructions may he in any form, including paper or digital. The instructions may be on the inside or the outside of the package. The instructions may be in the form of an internet address which provides the detailed manipulative or analytic techniques. The package may be divided so that components are not mixed until desired. Components may be in different physical states. For example, some components may be lyophilized and some in aqueous solution. Some may be frozen. Individual components may be separately packaged within the kit. The kit may contain reagents, as described above for sequencing the 16S rRNA V3-V5 regions. Desirably the kit will contain oligonucleotide primers which specifically hybridize to regions within 1 kb of the transcription start sites of the selected regions. Additional regions may be used. Typically the kit will contain both a forward and a reverse primer for a single region or marker. If there is a sufficient region of complementarity, e.g., 12, 15, 18, or 20 nucleotides, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The kit may also contain components for performing amplification, such as a DNA polymerase (particularly a thermostable DNA polymerase) and deoxyribonucleotides, labeled or not. Means of detection may also be provided in the kit, including detectable labels on primers or probes. Kits may also contain reagents for detecting gene expression. Such reagents may include probes, primers, or antibodies, for example. In the case of enzymes or ligands, substrates or binding partners may be used to assess the presence of the marker. Kits may contain 1, 2, 3, 4, or more of the primers or primer pairs of the invention. Kits that contain probes may have them as separate molecules or covalently linked to a primer for amplifying the region to which the probes hybridize. Other useful tools for performing the methods of the invention or associated testing, therapy, or calibration may also be included in the kits, including buffers, enzymes, gels, plates, detectable labels, vessels, etc. Kits may include tools for collecting suitable samples, such as tools for collecting oral swabs, oral biopsies, and endoscopes.

In addition, the kits may include instructional materials containing directions (e.g., protocols) for the practice of the methods described herein.

Method of diagnosis and differentiation are also provided. The methods comprise detecting at least one or more disease-associated bacterial species in a biological sample. The sample may be derived from a subject. Diagnosis of a disease state in a subject may allow for prognosis and selection of therapeutic strategy. Further, the developmental stage of cells may be classified by determining temporarily expressed disease-associated bacterial species.

Expression of a gene can be assessed using any means known in the art. Typically expression is assessed and compared in test samples and control samples which may be normal, non-malignant cells. The test samples may contain cancer cells or pre-cancer cells or nucleic acids from therm. Samples will desirably contain squamous cells. Samples may contain mixtures of different types and stages of cancer cells. Either mRNA (or cDNA) or protein can be measured to detect expression which may be used as an indicator of epigenetic modification. Methods employing hybridization to nucleic acid probes can be employed for measuring specific mRNAs. Such methods include using nucleic acid probe arrays (microarray technology), in situ hybridization, and using Northern blots. Messenger RNA can also be assessed using amplification techniques, such as RT-PCR. Advances in genomic technologies now permit the simultaneous analysis of thousands of genes, although many are based on the same concept of specific probe-target hybridization. Sequencing-based methods are an alternative; these methods may be based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and hands that differ between two tissues identify cDNAs of interest.

Exemplary biochips of the present invention include an organized assortment of oligonucleotide probes described above immobilized onto an appropriate platform. Each probe selectively binds a 16S rRNA V3-V5 region in a sample. In certain embodiments, each probe of the biochip selectively binds a biologically active 16S rRNA V3-V5 region in a sample.

In accordance with another embodiment, the biochip of the present invention can also include one or more positive or negative controls. For example, oligonucleotides with randomized sequences can be used as positive controls, indicating orientation of the biochip based on where they are placed on the biochip, and providing controls for the detection time of the biochip when it is used for detecting 16S rRNA V3-V5 regions in a sample.

Embodiments of the biochip can be made in the following manner. The oligonucleotide probes to be included in the biochip are selected and obtained. The probes can be selected, for example, based on a particular subset of 16S rRNA V3-V5 regions of interest. The probes can be synthesized using methods and materials known to those skilled in the art, or they can be synthesized by and obtained from a commercial source, such as GeneScript USA (Piscataway, N.J.).

Each discrete probe is then attached to an appropriate platform in a discrete location, to provide an organized array of probes. Appropriate platforms include membranes and glass slides. Appropriate membranes include, for example, nylon membranes and nitrocellulose membranes. The probes are attached to the platform using methods and materials known to those skilled in the art. Briefly, the probes can be attached to the platform by synthesizing the probes directly on the platform, or probe-spotting using a contact or non-contact printing system. Probe-spotting can be accomplished using any of several commercially available systems, such as the GeneMachines™ OmniGrid (San Carlos, Calif.).

The 16S rRNA V3-V5 regions in a sample can be amplified and labeled as is appropriate or desired. If amplification is desired, methods known to those skilled in the art can be applied. The samples can be labeled using various methods known to those skilled in the art. In accordance with an embodiment, the samples are labeled with digoxigenin using a Digoxigenin (DIG) Nucleotide Tailing Kit (Roche Diagnostics Corporation, Indianapolis, Ind.) in a GeneAmp® PCR System 9700 (Applied Biosystems, Foster City, Calif.).

The labeled 16S rRNA V3-V5 region sample is incubated with the biochip, allowing the miRNAs in the sample to hybridize with a probe specific for the 16S rRNA V3-V5 regions in the sample. In certain embodiments, the labeled 16S rRNA V3-V5 region sample is added to a DIG Easy Hyb Solution or Hybrid Easy Buffer (Roche Diagnostics Corporation, Indianapolis, Ind.) that has been preheated to hybridization temperature. The sample is the incubated with the biochip in the solution, for example, for about 4 hours to about 24 hours.

The 16S rRNA V3-V5 regions in the sample can be detected, identified, and quantified in the following manner. After the miRNA sample has been incubated with the biochip for an appropriate time period, the biochip is washed with a series of washing buffers, and then incubated with a blocking buffer. When Digoxigenin (DIG) labeling of the 16S rRNA V3-V5 region samples has been used, the biochip is then incubated with an Anti-DIG-AP antibody (Roche Diagnostics Corporation, Indianapolis, Ind.). The biochip is them washed with washing buffer and incubated with detection buffer, for example, for about 5 minutes. NBT/BCIP dye (5-Bromo-4-Chloro-3′-Indolyphosphate p-Toluidine Salt and NBT Nitro-Blue Tetrazolium Chloride) diluted with detection buffer is added to the biochip, which is allowed to develop in the dark, for example, for about 1 hour to about 2 days under humid conditions.

The biochips are scanned, for example, using an Epson Expression 1680 Scanner (Seiko Epson Corporation, Long Beach, Calif.) at a resolution of about 1500 dpi and 16-bit grayscale. The biochip images are analyzed using Array-Pro Analyzer (Media Cybernetics, Inc., Silver Spring, Md.) software. Because the identity of the miRNA probes on the biochip are known, the sample can be identified as including particular miRNAs when spots of hybridized miRNAs-and-probes are visualized. Additionally, the density of the spots can be obtained and used to quantitate the identified 16S rRNA V3-V5 region in the sample.

The identity and relative quantity of 16S rRNA V3-V5 regions in a sample can be used to provide a bacterial profile for a particular sample. For example, a bacterial profile for a sample includes information about the identities, quantitative levels, and/or changes in quantitative levels of 16S rRNA V3-V5 regions of bacterial species and associated a particular cellular type, process, condition of interest, or other cellular state. Such information can be used, for diagnostic purposes, drug development, drug screening and/or drug efficacy testing. In an embodiment, the bacterial species f the present invention are present in subjects having pre-clinical HNSCC. For example, the presence of these bacterial species in high levels compared with controls indicates a diagnosis of HNSCC in a subject.

In some embodiments, the 16S rRNA V3-V5 regions of the bacterial species can be identified using known next generation sequencing equipment such as the 454 FLX Titanium sequencer. It will be understood by those of ordinary skill in the art, that the genomes of the bacteria in the samples can be amplifed and analyzed using any amplification and sequencing methods currently available, with the specific primers and probes for the 16S rRNA sequence of the bacterial genome of interest.

Testing can be performed diagnostically or in conjunction with a therapeutic regimen for HNSCC or OPSCC. Examples standard treatment include radiation therapy, surgery, and chemotherapy. Some alternative treatment modalities are being tested in clinical trials and include targeted therapy and radiosensitizers.

Radiation therapy can be external, internal and hyperfractionated.

Chemotherapeutic agents used in HNSCC can include Abitrexate (Methotrexate), Blenoxane (Bleomycin), Cetuximab, Folex (Methotrexate), Folex PFS (Methotrexate), Hydroxyurea, Keytruda (Pembrolizumab), Methotrexate, Methotrexate LPF (Methotrexate), Opdivo (Nivolumab), Pembrolizumab, and Taxotere (Docetaxel).

Targeted therapy is a type of treatment that uses drugs or other substances to attack specific cancer cells. Targeted therapies usually cause less harm to normal cells than chemotherapy or radiation therapy do. Monoclonal antibodies are exemplary of one type of targeted therapy in the treatment of laryngeal cancer. Monoclonal antibody therapy is a cancer treatment that uses antibodies made in the laboratory from a single type of immune system cell. These antibodies can identify substances on cancer cells or normal substances in the blood or tissues that may help cancer cells grow. The antibodies attach to the substances and kill the cancer cells, block their growth, or keep them from spreading. Monoclonal antibodies are given by infusion. They may be used alone or to carry drugs, toxins, or radioactive material directly to cancer cells. Cetuximab is a type of monoclonal antibody that is being studied in the treatment of laryngeal cancer. It works by binding to a protein on the surface of the cancer cells and stops the cells from growing and dividing.

HPV/OPSCC patients are treated with standard chemoradiation therapy or surgery with a generally high response rate. However, treatment is often complicated by morbidities and the positive prognostic benefit of HPV can be mitigated by negative prognostic factors, such as smoking and lymph node metastases. Recurrence, metastasis, and second primary cancer still account for the majority of deaths from HPV/OPSCC. The appreciable number of deaths from recurrence or treatment failure (˜15 to 18/100, even with the most aggressive current treatment), coupled with a dramatic surge in total number of HPV/OPSCC suggests that the total number of deaths from both HPV-positive and HPV-negative OPSCCs will be comparable. More strikingly, a recent study revealed that the highest-risk HPV/OPSCC patients had a 3-year overall survival of only 70.8%, while the overall survival for the highest-risk HPV-negative OPSCC was 46%. It is therefore important to determine the most effective treatment strategy for HPV/OPSCC. Currently, there is no single standardized treatment for OPSCCs in the US. Surgery, chemotherapy, or radiation therapy, or combinations of these modalities have all been used at different centers in the country. Most importantly, adjuvant radiation (or chemoradiation) is often required following surgery, so patients treated primarily by surgical resection are still exposed to the side effects of these non-surgical approaches. In general, systemic therapy and radiation therapy are relatively more amenable to standardization across institutions and delivery on clinical protocols can be widely available in most cancer centers.

Testing can be used to monitor efficacy of a therapeutic regimen, for example, whether a chemotherapeutic agent or a biological agent, such as a polynucleotide. Testing can also be used to determine what therapeutic or preventive regimen to employ on a patient or for adjusting or modifying the planned treatment of the subject as a result of the increased risk of poor survival in the subject having head and neck squamous cell cancer.

Moreover, testing can he used to stratify patients into groups for testing agents and determining their efficacy on various groups of patients. Such uses characterize the cancer into categories based on the genes which are epigenetically silenced and/or the amount of silencing of the genes. In the case of a diagnosis or characterization, information comprising data or conclusions can be written or communicated electronically or orally. The identification may be assisted by a machine. Communication of the data or conclusions may be from a clinical laboratory to a clinical office, from a clinician to a patient, or from a specialist to a generalist, as examples. The form of communication of data or conclusions typically may involve a tangible medium or physical human acts.

In accordance with some embodiments, the present invention provides a method for triaging a HNSCC diagnosed subject into treatment.

Although diagnostic and prognostic accuracy and sensitivity may be achieved by using a combination of 16S rRNA V3-V5 regions, such as 3 or 4 regions, or 5 or 6 regions, practical considerations may dictate use of smaller combinations. Any combination of regions for a specific diagnosis may be used which comprises 2, 3, 4, or 5 regions. Combinations of 2, 3, 4, or 5 regions can be readily envisioned given the specific disclosures of individual regions provided herein.

In some embodiments, the presently disclosed subject matter further comprises recommending treatment and/or treating the subject. Treatment may include removal of precancerous lesions; radiation treatment; surgery, such as a laryngectomy, removal of lymph nodes, a biopsy, and/or chemotherapy. In some embodiments, the treatment is selected from the group consisting of removal of precancerous lesions, radiation treatment, surgery, and chemotherapy.

### EXAMPLES

DNA Extraction

Microdissected tissues and saliva (2 mL) samples were centrifuged and the pellets were digested with 1% SDS and 50 μg/mL proteinase K (Boehringer, Mannheim, Germany) at 48° C. overnight extracted with phenol/chloroform, precipitated in 100% ethanol, centrifuged at 5100 rpm for 45 minutes, washed in 70% ethanol twice, dissolved in LoTE buffer (10 mM TRIS hydrochloride, 1 mM EDTA buffer, pH 8), and stored at −20° C. [48].

Quantitative PCR

The 7900HT real time PCR system was used to perform quantitative PCR for HPV-16 E6 and E7 and B-actin. Specific primers and probes have been designed to amplify the E6 and E7 regions of HPV type 16: HPV-16 E6 forward primer, 5′-TCAGGACCCACAGGAGCG-3′ (SEQ ID NO: 1); HPV-16 E6 reverse primer, 5′-CCTCACGTCGCAGTAACTGTTG-3′ (SEQ ID NO: 2), HPV-16 E6 TaqMan probe, 5′-(FAM)-CCCAGAAAGTTACCACAGTTATGCACAGAGCT-(TAMRA)-3′ (SEQ ID NO: 3), HPV-16 E7 forward primer, 5′-CCGGACAGAGCCCATTACAA-3′ (SEQ ID NO: 4), HPV-16 E7 reverse primer, 5′-CGAATGTCTACGTGTGTGCTTTG-3′ (SEQ ID NO: 5), HPV-16 E7 TaqMan probe, 5′-(FAM)-CGCACAACCGAAGCGTAGAGTCACACT-(TAMRA)-3′ (SEQ ID NO: 6). A housekeeping gene (B-globin) were run in parallel with HPV-16 E6 and E7 to standardize the input DNA: B-actin forward primer, 5′-TCACCCACACTGTGCCCATCTACGA-3′ (SEQ ID NO: 7), B-actin reverse primer, 5′-CAGCGGAACCGCTCATTGCCAATGG-3′ (SEQ ID NO: 8), B-actin TaqMan probe, 5′-(FAM)-ATGCCCTCCCCCATGCCATCCTGCGT-(TAMRA)-3′ (SEQ ID NO: 9). All samples were run in triplicate.

HPV Data Analysis

The CaSki (American Type Culture Collection, Manassas, Va.) cell line was used to develop standard curves for the HPV viral copy number as it is known to have 600 copies/genome equivalent. Standard curves for HPV-16 E6 and E7 were developed by using DNA extracted from CaSki cells, serially diluted into 50 ng, 5 ng, 0.5 ng, 0.05 ng, and 0.005 ng. A standard curve was also developed for the housekeeping gene B-actin (2 copies/genome), using the same serial dilutions of CaSki DNA. Tumor samples with >0.1 copy/genome and salivary samples with >0 copy/genome were considered as HPV positive. Simple sensitivity and specificity analyses were performed on the cases with local recurrence. No statistical correlation was attempted due to modest sample size.

Creation of the 16S rRNA V3-V5 Amplicon Library

An amplicon library from individual samples was created by PCR amplification with unique barcoded primers of the 16S rRNA V3-V5 gene region, using the 357F/926R primer set. We used 14 different barcode sequences and the linker primer sequence CCGTCAATTCMTTTRAGT (SEQ ID NO: 10) to analyze the 16S rRNA V3-V5 hypervariable 16S rRNA gene region.

DNA Sequencing and Bioinformatics Analyses

Sequencing of the multiplexed amplified fragments was performed on the Roche/454 GS Junior pyrosequencing platform. Bioinformatics preprocessing steps included quality filtering, error-correction, and chimera removal. Briefly, reads were de-multiplexed using 5′ barcodes, trimmed of forward and reverse primer sequences, filtered for length and quality, and corrected for homopolymer errors. High quality reads were selected for analysis and reads with unknown bases (“N”) were discarded. The resulting high-quality dataset was then screened for chimeric sequences and contaminant chloroplast DNA.

Passing sequences were characterized for diversity and taxonomic composition using the Quantitative Insights into Microbial Ecology (QIIME) suite, version 1.9 where all the beta and alpha diversity measure and significance tests were performed [49, 50]. To begin, sequences were clustered into operational taxonomic units (OTUs) using UCLUST with a 97% identity threshold. Taxonomic assignment was performed using the RDP classifier (trained by a customized version of the comprehensive GreenGenes database, release v.13-05) with a minimum confidence threshold of 0.80. After considering the raw count data in full above, subsample analysis of each community was performed to an equivalent depth, in this case, 3,400 sequences per sample. All results are based on the subsampled data, which mitigates biases due to differences in sampling depth.

An OTU network was generated using QIIME [50] and imported to Cytoscape [51] based on OTUs that changed significantly in abundance (p<0.05) as result of a maximum likelihood statistical significance tests. The selected OTUs were plotted choosing nodes from the OTU network and sorting edges interaction by the four different sample types: normal, HPV negative OSCC, HPV negative OPSCC and HPV positive OPSCC. Additionally, we represented the taxonomy of taxa at the genus-level through pie charts at each of the four sample types.

Species Level Identification with Resphera Insight

High-quality 16S rRNA amplicon sequences passing preprocessing were submitted to Resphera Insight (Baltimore, Md., www.respherabio.com) for high-resolution taxonomic identification [52-55]. The Insight protocol is designed to provide accurate species-level context for 16S rRNA microbiome profiling studies, and has been benchmarked on 540 pathogens listed by the CDC National Healthcare Safety Network (NHSN). After taxonomic assignment, the dataset was rarefied to 3,400 sequences per sample to provide an even level of coverage for downstream statistical comparisons.

As a validation set, we obtained preprocessed 16S amplicon datasets (V3V5 region sequenced using Roche/454 platform) from 537 unique samples reflecting healthy human saliva, mid-vagina, and vaginal introitus environments from the Human Microbiome Project Data Analysis and Coordination Center (http://hmpdacc.org) [56, 28]. After filtering of trimmed sequences less than 200 bp and requiring a minimum of 2,500 sequences for analysis, 514 of the 537 samples were subsequently analyzed using Resphera Insight: healthy human saliva (n=265), mid-vagina (n=128), and vaginal introitus (n=121). These samples were obtained from 154 unique participants: saliva (n=154), mid-vagina (n=79), and vaginal introitus (n=73), most of whom provided two longitudinal samples.

Statistical Analysis

For each group comparison, significance tests were computed including the maximum likelihood statistical significance tests that determine whether OTU presence/absence is associated with a category in the metadata. The goodness of fit or log-likelihood ratio parametric test (G-test) compares the ratio of the observed OTU frequencies in the sample groups to the expected frequencies based on the null hypothesis (all sample groups have equal OTU frequencies). QIIME [50] was used to create all the heatmaps and estimate the following Alpha-diversity metrics: raw number of OTUs per sample, Chaol estimator, Shannon entropy, Non-Metric dimensional scaling, and Bray-Curtis distance metric.

The chao 1 index approach for richness was used because it uses the numbers of singletons (OTUs with single appearance) and doubletons (OTUs that appeared twice) to estimate the number of missing species because missing species information is mostly concentrated on low frequency counts. Faith's phylogenetic diversity index (PD) estimates the relative feature diversity of any nominated set of species by the sum of the lengths of all phylogenetic branches required to span a given set of taxa on the phylogenetic tree. The relative group variance homogeneity was verified with the function ‘betadisper’ also in the “vegan” package. Richness box and whisker plots were calculated using both vegan [57] and Phyloseq [58] R packages.

We used linear discriminant analysis (LDA) with LefSe [59] an algorithm biomarker discovery that identifies taxa characterizing the differences between two metadata classes. It emphasizes statistical significance, biological consistency and effect relevance, allowing researchers to identify differentially abundant features that are also consistent with biologically meaningful categories (metadata), using non-parametric factorial Kruskal-Wallis (KW) sum-rank test, Wilcoxon rank-sum test and LDA. High LDA scores reflect significantly higher abundance of certain taxa.

### Example 1

Discovery Cohort Patients from Johns Hopkins Hospital

The Discovery cohort patients selected for this study were nested within a longitudinal cohort study of 787 patients who presented with histopathologically confirmed HSNCC (this includes patients who presented for treatment of a recurrence after primary treatment at an outside hospital) to the outpatient clinic of the Johns Hopkins Hospital in Baltimore, Md. from 2000 to 2011. Patients were included if they had at least one pre-treatment salivary sample and consented for the study. All patients had undergone treatment with curative intent. The study protocol was approved by the institutional review board of the Johns Hopkins Hospital, as well as by the Johns Hopkins Institutional Review Board. Written informed consent was obtained from all patients.

Patients were consented for this study under the molecular surveillance clinical research protocol. Saliva was collected from 44 patients: 25 patients with no history of cancer and 19 HNSCC patients. Longitudinal saliva samples were collected from 58% of the HNSCC patients, totaling 62 samples. Nonetheless, we eliminated 3 samples from 2 patients (2 OSCC and 1 OPSCC), 2 of them with unknown HPV status, as well as the only HPV positive OSCC sample. The analyses presented here are based on a total of 59 saliva samples acquired from 42 patients; of these, 34 saliva samples corresponded to 17 HNSCC patients and 25 saliva samples corresponded to 25 controls without cancer, which also had negative smoking and drinking histories. Most of the HNSCC patients were OPSCC patients (7 were HPV positive and 4 were HPV negative), and the rest were OPSCC patients (all 6 were HPV negative) (Table 1).

TABLE 1
Number of Johns Hopkins Hospital patients, number of samples, total
number of sequences and total number of significant Operational Taxonomic Units by
histology type and HPV status.

| | Number of | Number of | Total Number of | Total Number of |
|---|---|---|---|---|
| Sample type (Histology + HPV status) | Patients | Samples | Sequences | OTUs |
| Normal Mucosa (Control) HPV | 25 | 25 | 31,196 | 67 |
| Negative | | | | |
| Oropharynx Squamous cell carcinoma | 4 | 11 | 7,192 | 47 |
| (OPSCC) HPV Negative | | | | |
| Oropharynx Squamous cell carcinoma | 7 | 13 | 7,278 | 41 |
| (OPSCC) HPV Positive | | | | |
| Oral Cavity Squamous cell carcinoma | 6 | 10 | 4,828 | 55 |
| (OSCC) HPV Negative | | | | |
| Grand Total | 42 | 59 | 50,494 | 210 |

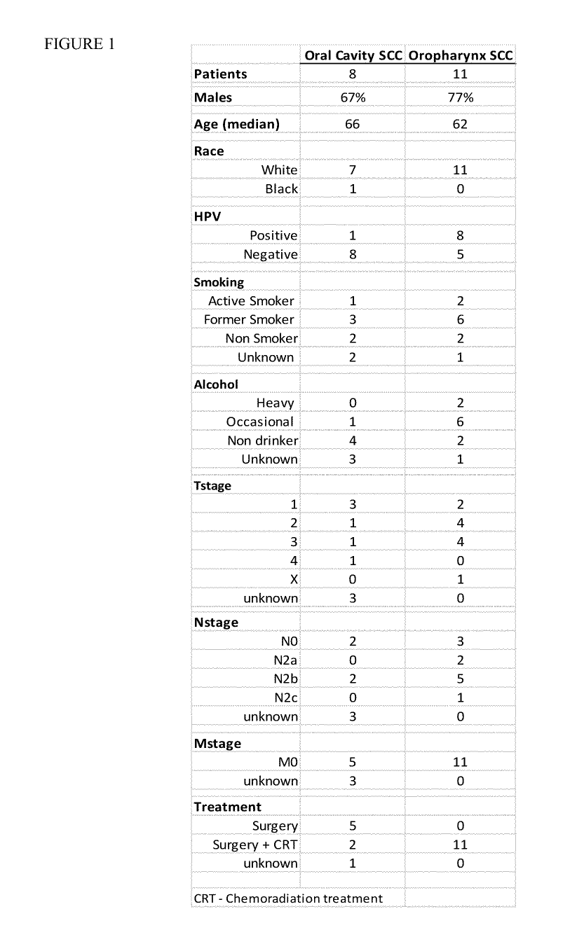

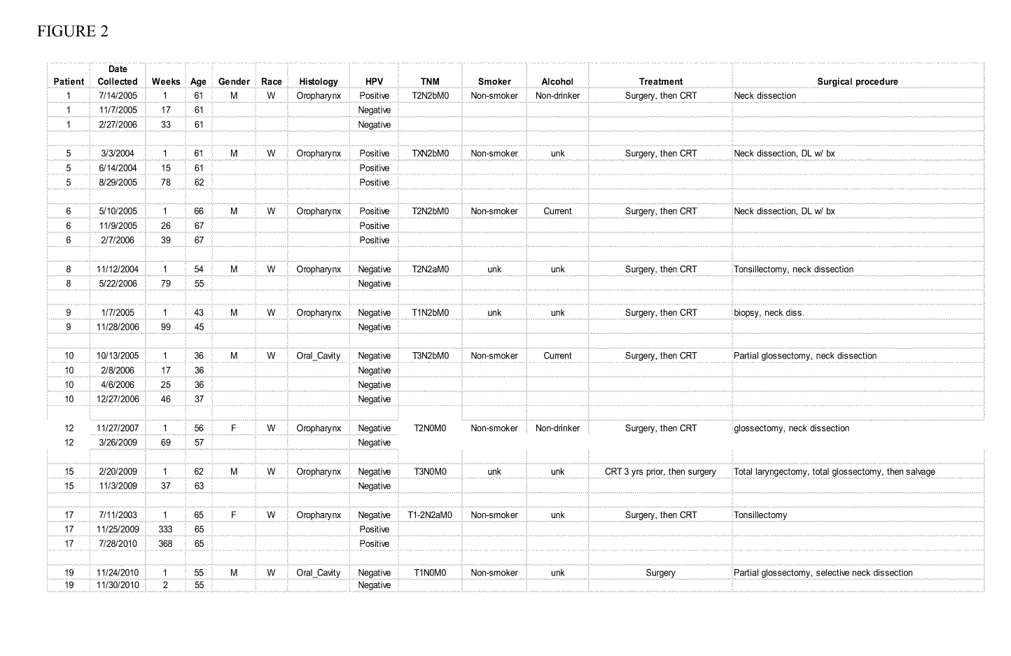

Tissue and saliva samples were collected between 2000 and 2011 and stored in the Johns Hopkins Head and Neck Cancer Research Division's Tumor Bank, from where they were randomly selected for this study (See Table at FIG. 1). Fifty-eight percent (58%) of the HNSCC patients chosen for the study had OPSCC. There was no difference in median age (66 and 62 years) or percentage of male patients (67% and 77%) between OSCC and OPSCC patients. Forty-seven percent (47%) of the HNSCC patients were HPV positive. Most HNSCC patients were former smokers or never smokers: OPSCC (72%) and OSCC (63%). Similarly, most HNSCC patients were occasional drinkers or non-drinkers: OPSCC (72%) and OSCC (63%). Approximately half of the patients were diagnosed with T1 or T2 stage tumors: OSCC (50%) and OPSCC (55%). Most patients had nodal involvement: OSCC (75%) and OPSCC (73%). None of the patients had known metastasis. All the OPSCC were treated with surgery and chemoradiation, compared to only 25% of the OSCC. Most of the OSCC patients (63%) only required surgical treatment. (See Table at FIG. 2).

We acquired a set of 607,646 raw reads when we sequenced the 16S rRNA V3-V5 hypervariable region from the 62 DNA samples used in the study using the 454 FLX Titanium sequencer. Sequences underwent strict quality and size filtering, removing reads shorter than 150 bp, as well as those with mismatches and poor quality scores. Sequences were then error-corrected using the Acacia tool, followed by de novo chimera detection with the UCHIME program, and screening for chloroplast contaminant sequences, as previously described[47]. Raw 16S rRNA sequences were collected and analyzed with Resphera Insight, a high-resolution methodology for 16S rRNA taxonomic assignment that is able to provide accurate species-level characterization. In total, 200,600 sequences passed preprocessing. The 200,600 sequences were binned into 1,504 OTUs. Control samples had the highest number of sequences and unique OTUs (85,000 and 507 respectively) while squamous cell carcinoma samples varied from 34,000 to 44,200 sequences and 318 to 356 OTUs respectively.

### Example 2

Validation Cohort from Human Microbiome Project

The Validation cohort samples selected for this study were nested within the 5,298 samples collected by the Human Microbiome Project (HMP) from a population of 242 healthy adults sampled at 15 or 18 body sites up to three times. Investigations of the microbiome from this cohort incorporated several complementary analyses including: 16S ribosomal RNA (rRNA) gene sequence (16S) and taxonomic profiles, whole-genome shotgun (WGS) or metagenomic sequencing of whole community DNA, and alignment of the assembled sequences to the reference microbial genomes from the human body. A total of 5,177 microbial taxonomic profiles from 16S-rRNA genes were characterized from habitats within the human airways, skin, oral cavity, gut and vagina.

As part of a multi-institutional collaboration, the HMP human subjects study was reviewed by the Institutional Review Boards (IRBs) at each sampling site: the BCM (IRB protocols H-22895 (IRB no. 00001021) and H-22035 (IRB no. 00002649)); Washington University School of Medicine (IRB protocol HMP-07-001 (IRB no. 201105198)); and St Louis University (IRB no. 15778). The study was also reviewed by the J. Craig Venter Institute under IRB protocol 2008-084 (IRB no. 00003721), and at the Broad Institute of MIT and Harvard the study was determined to be exempt from IRB review. All study participants gave their written informed consent before sampling and the study was conducted using the Human Microbiome Project Core Sampling Protocol A. Each IRB has a federal-wide assurance and follows the regulations established in 45 CFR Part 46. The study was conducted in accordance with the ethical principles expressed in the Declaration of Helsinki and the requirements of applicable federal regulations.

A total of 292 HMP saliva samples from 197 participants were selected for the Validation cohort. Sequences underwent strict quality and size filtering, removing reads shorter than 150 bp, as well as those with mismatches and poor quality scores. Sequences were then error-corrected using the Acacia tool, followed by de novo chimera detection with the UCHIME program, and screening for chloroplast contaminant sequences. In total, 252,764 sequences passed preprocessing. The 252,764 sequences were binned into 67 OTU.

A total of 128 HMP Mid Vagina samples from 96 participants were selected for the Validation cohort. Sequences underwent strict quality and size filtering, removing reads shorter than 150 bp, as well as those with mismatches and poor quality scores. Sequences were then error-corrected using the Acacia tool, followed by de novo chimera detection with the UCHIME program, and screening for chloroplast contaminant sequences. In total, 17,654 sequences passed preprocessing. The 17,654 sequences were binned into 35 OTUs.

A total of 121 HMP vaginal introitus samples from 90 participants were selected for the Validation cohort. Sequences underwent strict quality and size filtering, removing reads shorter than 150 bp, as well as those with mismatches and poor quality scores. Sequences were then error-corrected using the Acacia tool, followed by de novo chimera detection with the UCHIME program, and screening for chloroplast contaminant sequences. In total, 17,989 sequences passed preprocessing. The 17,989 sequences were binned into 31 OTUs.

### Example 3

Phyla and Species Levels Analyses

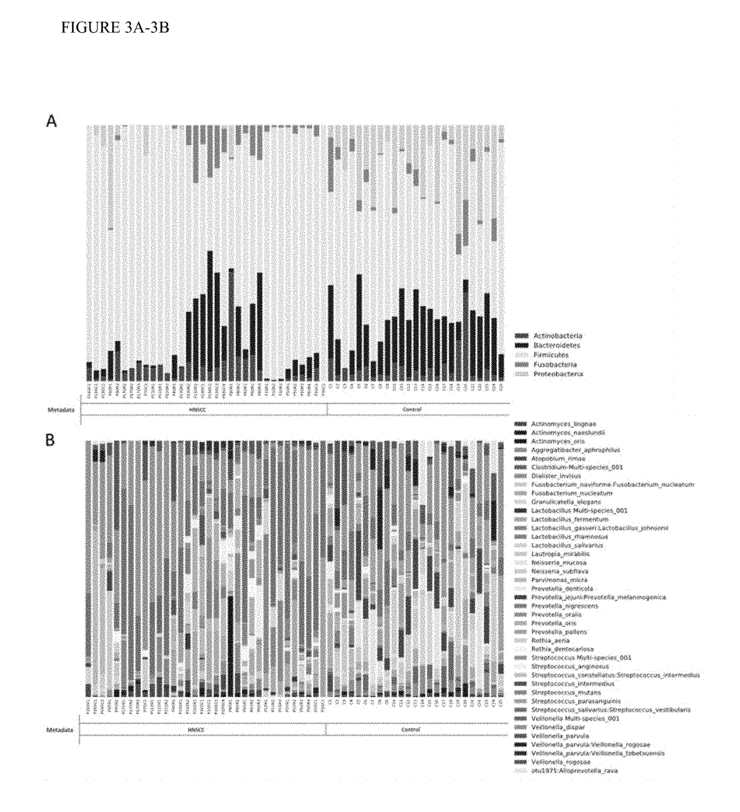

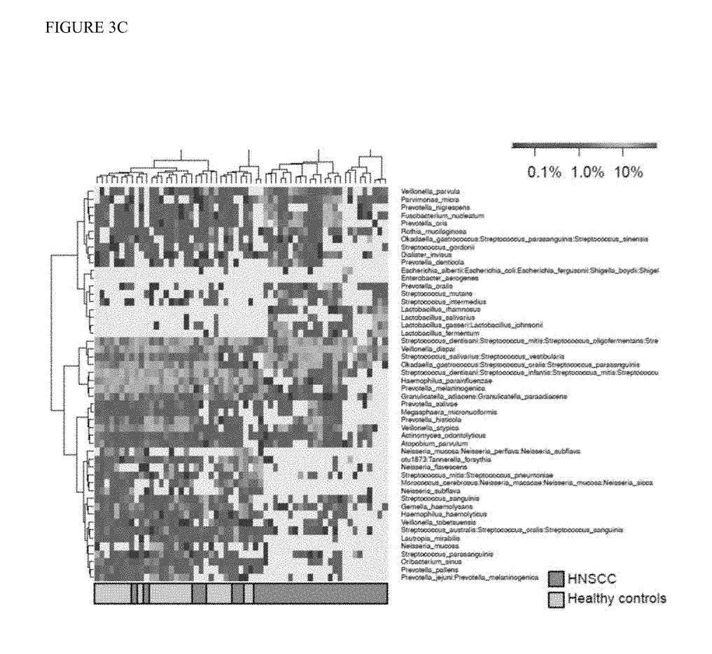

We found a total of 5 assigned phyla dominating across all of the samples: Actinobacteria, Bacteroidetes, Firmicutes, Fusobacteria, and Proteobacteria. (FIG. 3A). Overall, species-level profiles showed a dominance of Veilonella dispar across all samples (FIG. 3B). Streptococcus salivarius:Streptococcus vestibularis OTUs were more dominant in HNSCC samples Interestingly, at the species level we found Lactobacillus_gasseri: Lactobacillus_johnsonni, a microbial species only previously found in vagina, in saliva samples from a subset of OPSCC patients. The distinct presence of species in the saliva of HNSCC patients normally associated with the vaginal microbiome, most notably Lactobacillus gasseri/Lactobacillus johnsonii, which was 710× higher in HNSCC compared to controls in the Hopkins cohort, and Lactobacillus vaginalis, which was 52× higher in HNSCC compared to controls (p<0.05)

Hierarchical clustering of taxonomic profiles (top 50 species level calls) shows that the salivary microbiome is distinct in HNSCC, with significant enrichment of Lactobacillus spp, Parvimonas micra, Streptococcus mutans, and Fusobacterium nucleatum (p<0.05) in HNSCC samples and overall reduced alpha diversity (p<1e-4) (FIG. 3C). We also found a larger percentage of Fusobacterium_nucleatum present in a subset of HNSCC saliva samples when compared with controls. Remarkably there is a highly significant depletion of Fusobacterium periodonticum, Leptotrichia trevisanii, Leptotrichia hofstadii, and Leptotrichia buccalis in HNSCC compared to controls in the Hopkins cohort. Fusobacterium_nucleatum is closely related to each of these other species. Given these unexpected findings we decided to validate our results using the following samples from the Human Microbiome Project (HMP): saliva, vaginal-introitus and mid-vagina.

### Example 4

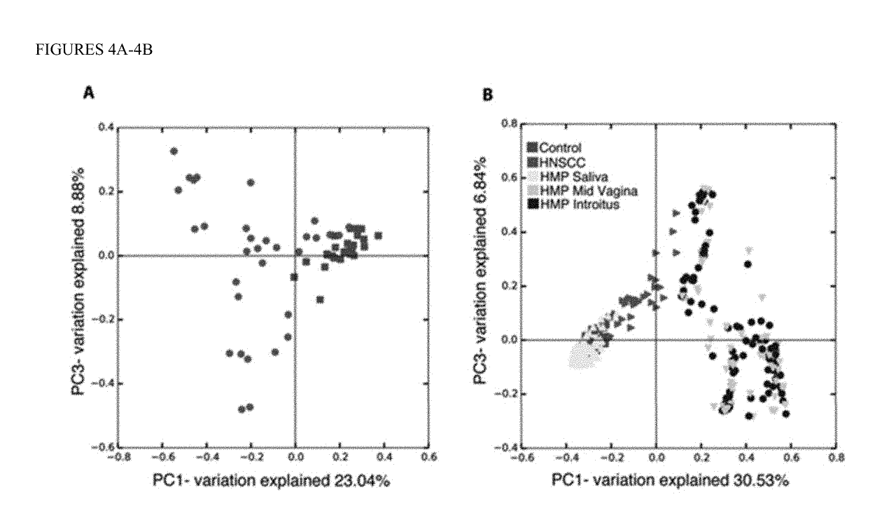

Principal coordinates analysis (PCoA) of the weighted and unweighted Unifrac distances (β-diversity) show differences in community structure in HNSCC when compared by normal samples (FIG. 4A). The 17 patient samples analyzed here corresponded only to the patient's first visit only. Principal coordinates analysis (PCoA) of the weighted and unweighted Unifrac distances (β-diversity) also show differences in community structure when comparing Hopkins HNSCC and normal samples from the HMP (FIG. 4B). We found dense clustering between HMP saliva, control samples and some tumor samples separated by principal component one, from those of HMP mid vagina and introitus in the coupled HNSCC+HMP sample collection.

### Example 5

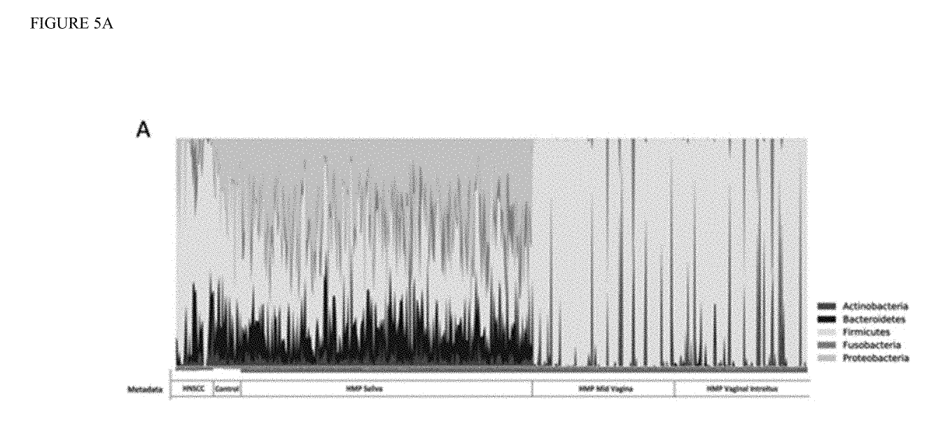

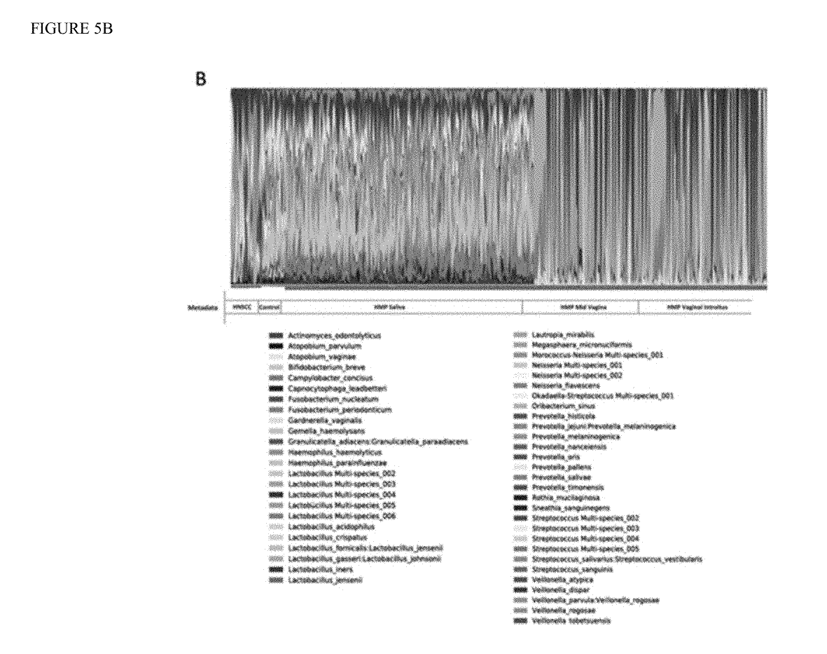

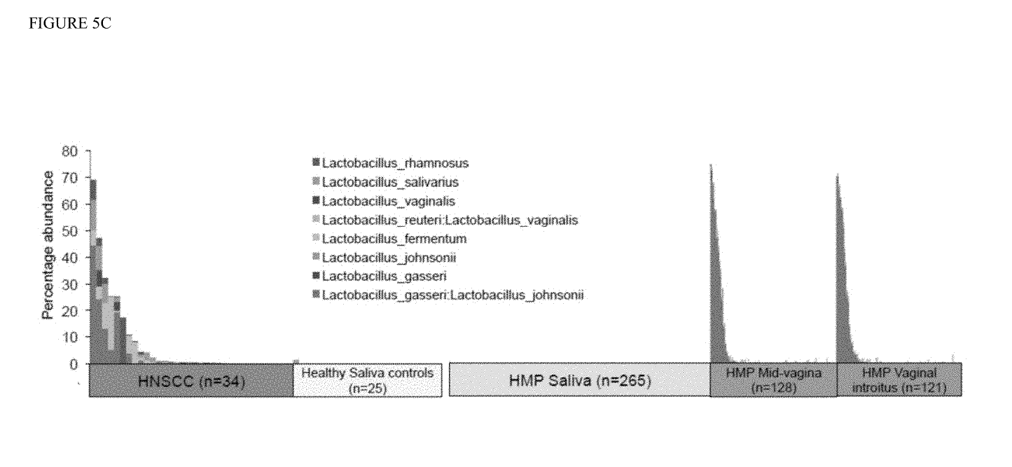

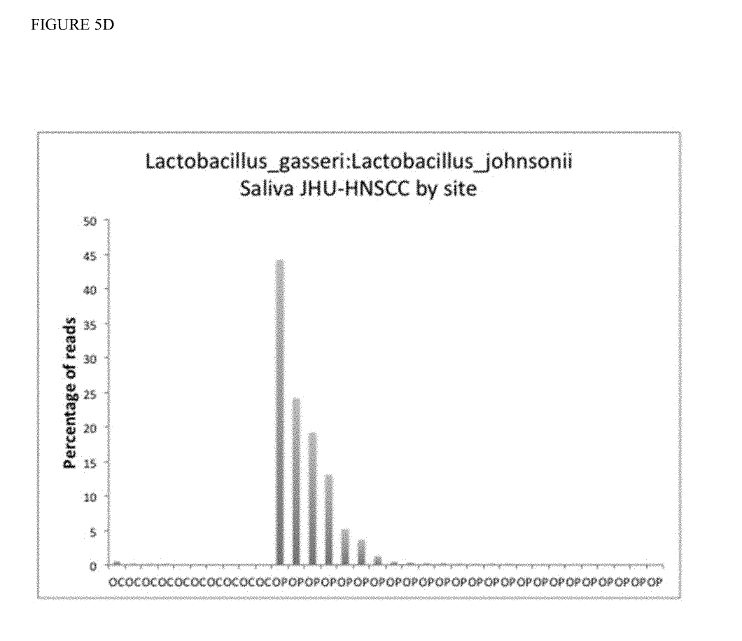

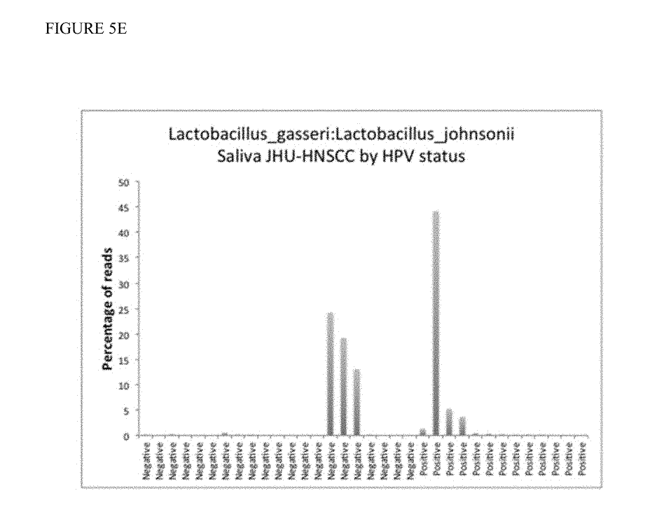

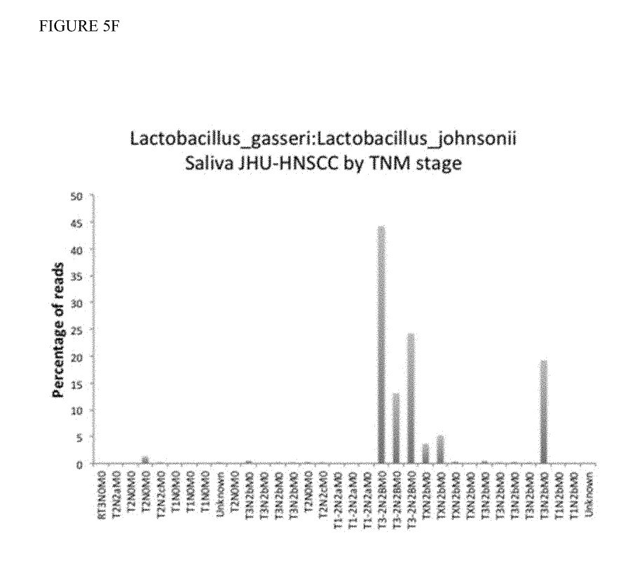

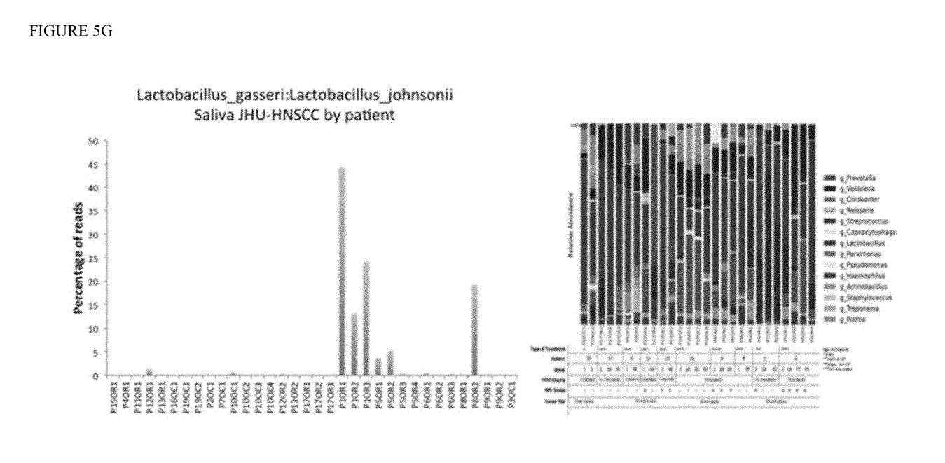

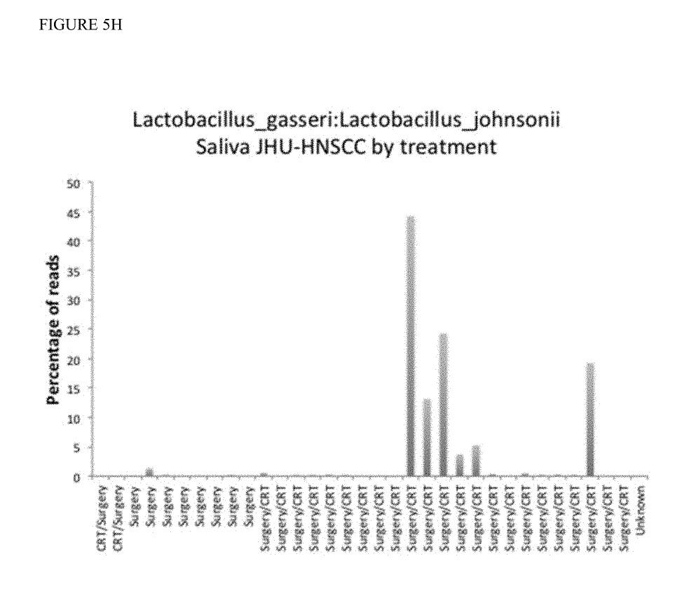

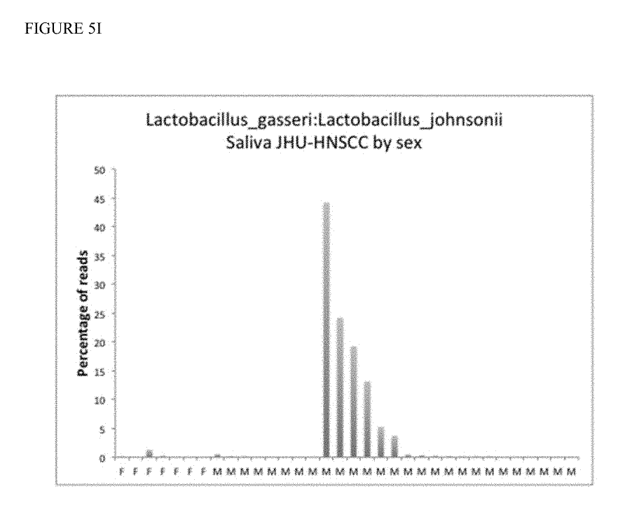

There were no differences in the microbial phyla present in the saliva control samples from Hopkins and the HMP after performing hierarchical clustering (FIG. 5A). The following microbial species clustered differently when comparing the samples from Hopkins and the HMP project Lactobacillus_gasseri: Lactobacillus_johnsonii was present in vaginal_introitus, mid_vagina samples from the HMP and in a subset of HNSCC saliva samples from Hopkins, but not in normal saliva samples from Hopkins or the HMP (FIG. 5B). An analysis of the percentage of significant reads for these cohorts also revealed that Lactobacillus_gasserii, Lactobacillus_johnsonii, Lactobacillus fornicalis, Lactobacillus jensenii, were present in vaginal_introitus, mid_vagina samples from the HMP, where they form part of the normal vaginal flora, and in a subset of HNSCC saliva samples from Hopkins, but not in HMP or Hopkins saliva control samples (FIG. 5C). The significant Lactobacillus_gasseri, and Lactobacillus_johnsonii reads were observed in 19 saliva samples from HNSCC patients: 15 samples (79%) from oropharyngeal cancer patients and 4 samples (21%) from oral cancer patients (FIG. 5D); 10 samples (53%) were from HPV positive saliva samples and 9 were from HPV negative samples (47%) (FIG. 5E); 17 (89%) of the saliva samples were from patients diagnosed with Stage IVa or higher HNSCC, with cancer spreading to at least 2 or more lymph nodes on the same side as the primary tumor, but none larger than 6 cm across, 1 saliva sample came from a patient who did not have nodal involvement and staging information was not available for one of the saliva samples (FIG. 5F); 17 (89%) came from 7 of the patients from whom we analyzed longitudinal saliva samples. Lactobacillus_gasseri, and Lactobacillus_johnsonii reads were observed in all 7 patients, before and after surgery (FIG. 5G). Most of the saliva samples (89%) with significant Lactobacillus_gasseri, and Lactobacillus_johnsonii reads were from patients treated with surgery and chemoradiation treatment (FIG. 5H). Only 2 (21%) of the samples came from patients treated with surgery alone. Most of the saliva samples were from male HNSCC patients (84%) (FIG. 5I).

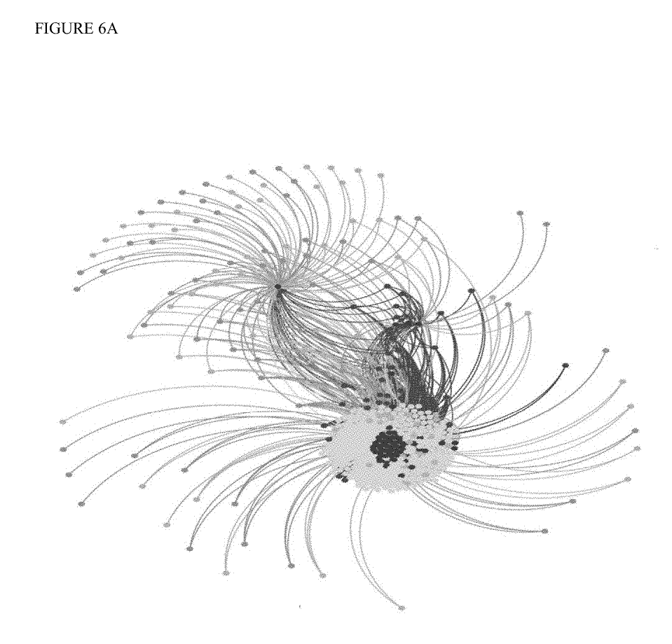

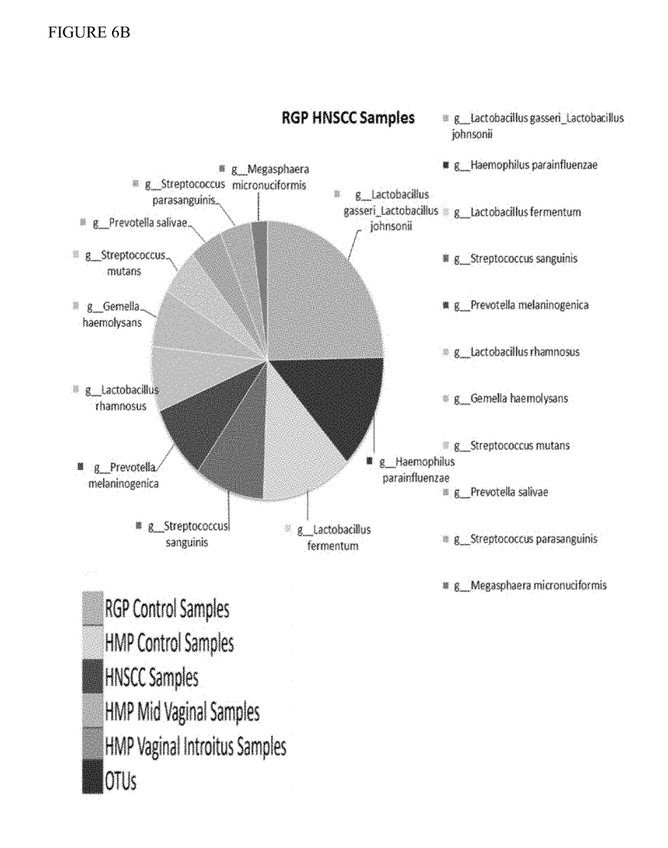

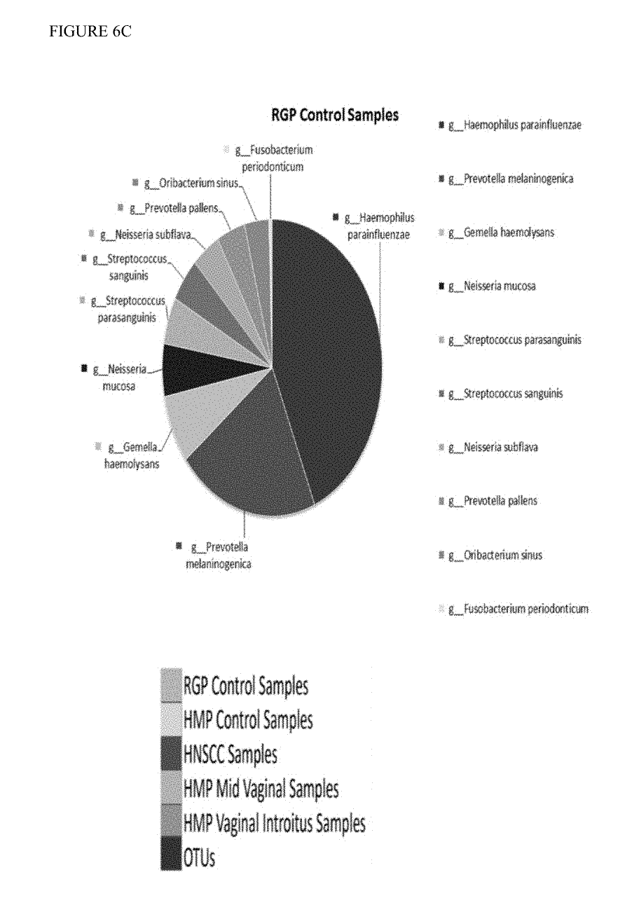

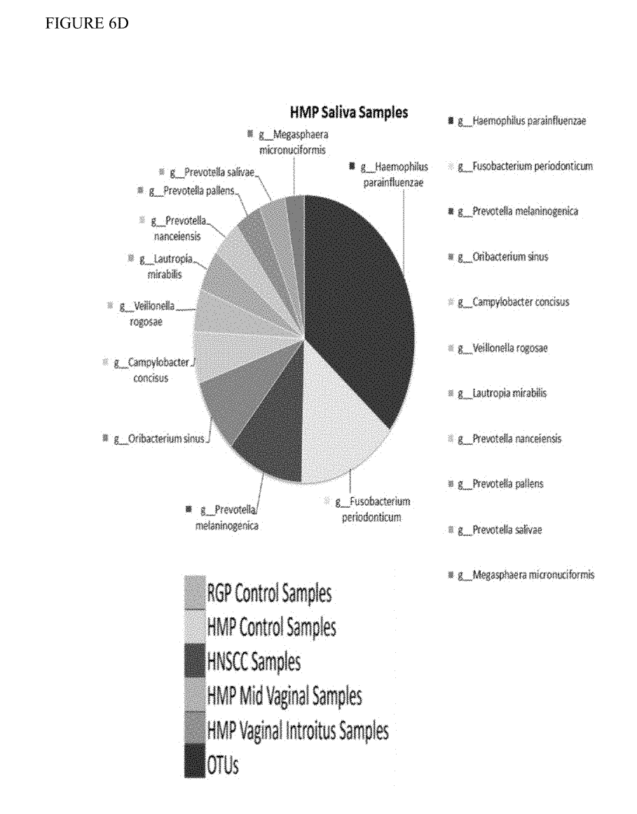

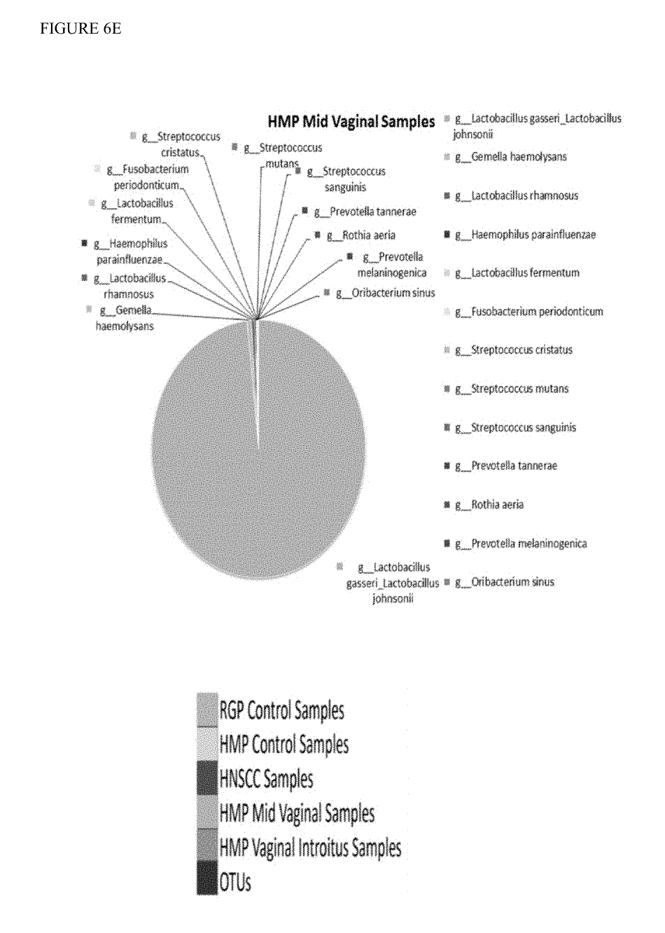

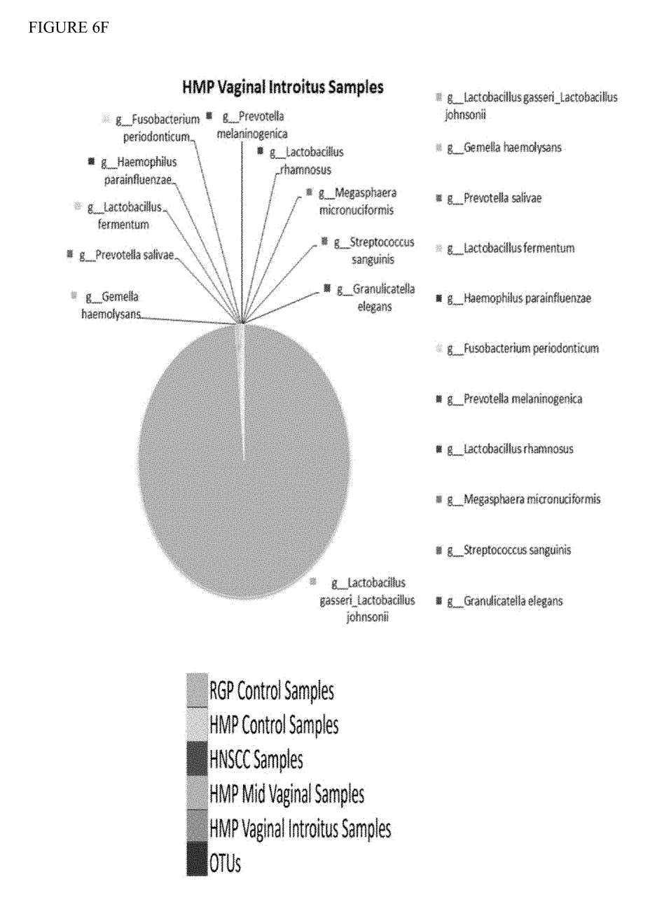

FIG. 6 represent species that differed significantly in relative abundance (p<0.05) when comparing saliva from normal patients with patients with HNSCC, normal saliva and vaginal flora samples from HMP. The figure was created using QIIME and imported to Cytoscape. Significant OTUs were obtained from an ANOVA test p<0.05 were selected and plotted in the OTU Network. Network layout was performed utilizing AllegroLayout a high-speed and high-quality interactive layout for large-scale networks using as parameter an OpenCL-accelerated force-directed layout edge-weighting algorithm. Result for this algorithm shows various clustering in according to metadata for HNSCC, RGP Control Samples, HMP Saliva Samples, HMP Mid Vaginal Samples and HMP Vaginal Introitus Samples. Pie charts were created showcasing taxa distinguishing samples by Histology and recruitment site. The OTU network (FIG. 6A) and pie charts shows the total abundance of species in saliva from HNSCC patients (RGP HNSCC—FIG. 6B), saliva from JHU normal participants (RGP Control—FIG. 6C), HMP saliva (FIG. 6D), as well as microbiome samples from mid vagina (FIG. 6E) and the vaginal introitus (FIG. 6F). Lactobacillus gasseri/johnsonii, Haemophilus parainfluenza, Lactobacillus fermentum and Fusobacterium periodonticum can be used to discriminate HNSCC samples from control samples. Similarly, samples for HNSCC, HMP Mid Vaginal and HMP Vaginal Introitus samples have a high abundance of Lactobacillus gasseri/johnsonii this similarity could suggest a correlation for HNSCC status for bacterial species obtained by some sort of interaction or vaginal exposure for patients with HNSCC status.

FIG. 6C was created using QIIME and imported to Cytoscape. Significant OTUs obtained from an ANOVA test p<0.05 were selected and plotted in the OTU Network. Network layout was performed utilizing AllegroLayout a high-speed and high-quality interactive layout for large-scale networks using as parameter an OpenCL-accelerated force-directed layout edge-weighting algorithm. Result for this algorithm shows various clustering in according to metadata for HNSCC samples (RGP HNSCC), JHU Normal samples (RGP Control), HMP Saliva Samples, HMP Mid Vaginal Samples and HMP Vaginal Introitus Samples. Pie charts were created showcasing taxa distinguishing samples by Tumor Histology and HPV status.

### Example 6

To better understand the OTU diversity in our cohorts, we compared the alpha rarefaction curves between normal and HNSCC sample categories according to the Chao 1 richness estimator (see methods). The chaol index estimates total species richness based on all species actually discovered, including species not present in any sample. This approach uses the numbers of singletons (single appearance) and doubletons (that appeared twice) to estimate the number of missing species due to undetected species information is mostly concentrated on low frequency counts.

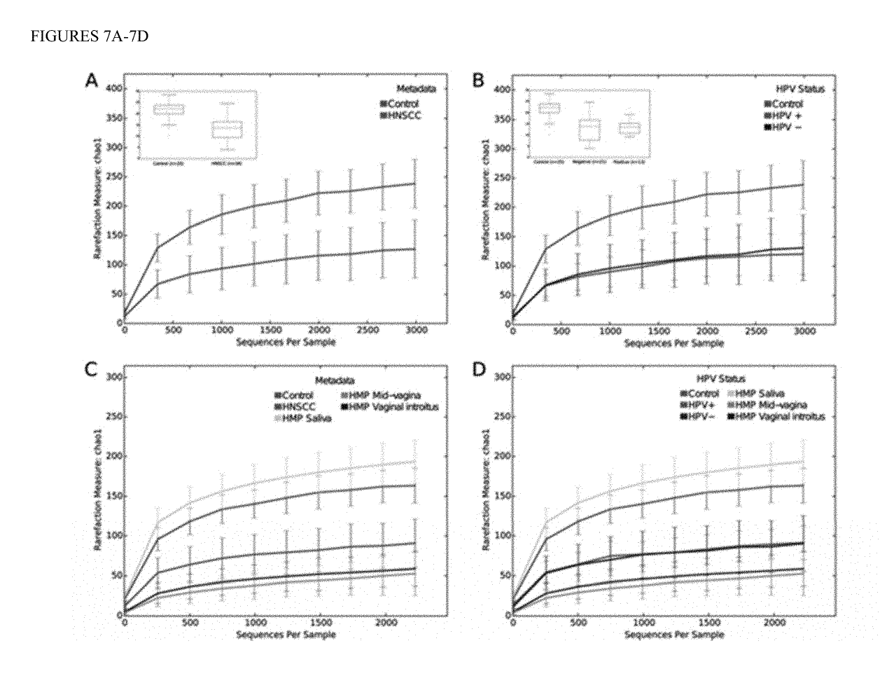

Microbial communities from control samples display significantly higher species richness (p<0.001) than HNSCC samples (FIG. 7A) regardless of whether they are HPV positive or HPV negative samples. (FIG. 7B). When examining Control and HNSCC samples with HMP saliva and vaginal samples, we found that Control and HMP saliva samples had the highest richness index followed by HNSCC and HMP vaginal samples (FIG. 7C). No significant differences were observed (p=0.15) in the HNSCC and HMP sample collection (FIG. 7D).

### Example 7

Differentially enriched Lactobacillus species OTUs in HNSCC and control samples were compared using the variance stabilization method of QIIME's 1.9.1 and DESeq2 normalization for data after logarithmic transformation, to provide a representation of differential abundance of Lactobacillus species between controls and HNSCC samples (data not shown). We found a significant association between Lactobacillus gasseri:johnsonni, L. fermentum and L. rhamnosus with HNSCC samples (p<0.0001). A single Lactobacillus OTU with multiple ambiguous Resphera assignments (multi species 008) was more abundant in control samples.

We similarly compared differentially enriched Lactobacillus species OTUs between control and HNSCC samples from Hopkins and HMP saliva, mid vagina and introitus (data not shown). This complex comparison revealed that species such as L gasseri, L. johnsonni, L. vaginalis, L. fermentum, L. salivarius and L. rhamnosus were differentially abundant across all the samples with higher association to HNSCC samples.

### Example 8

Differentially enriched Fusobacterium species OTUs in HNSCC and control samples were compared using the DESeq2 negative binomial Wald test for dispersion to provide a representation of differential abundance of Fusobacterium species between controls and a subset of HNSCC samples (data not shown). We found a significant association of Fusobacterium nucleatum and Fusobacterium naviforme in a subset of HNSCC samples. Fusobacterium species, such as F. canifelium, F. nucleatum and F. naviforme were differentially abundant across all samples.

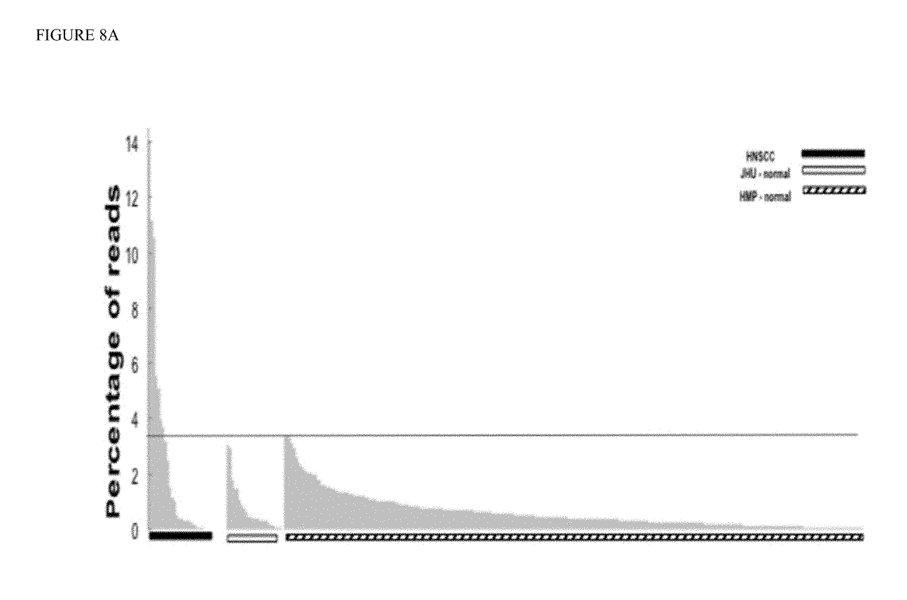

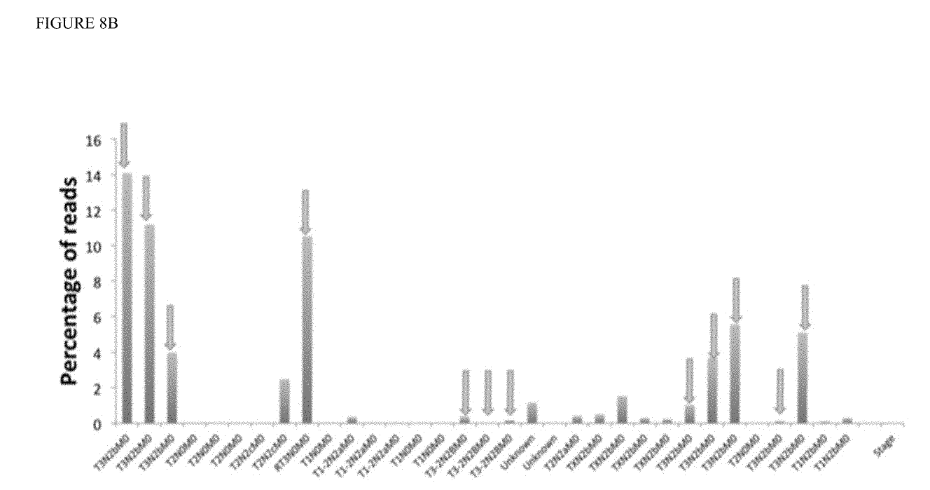

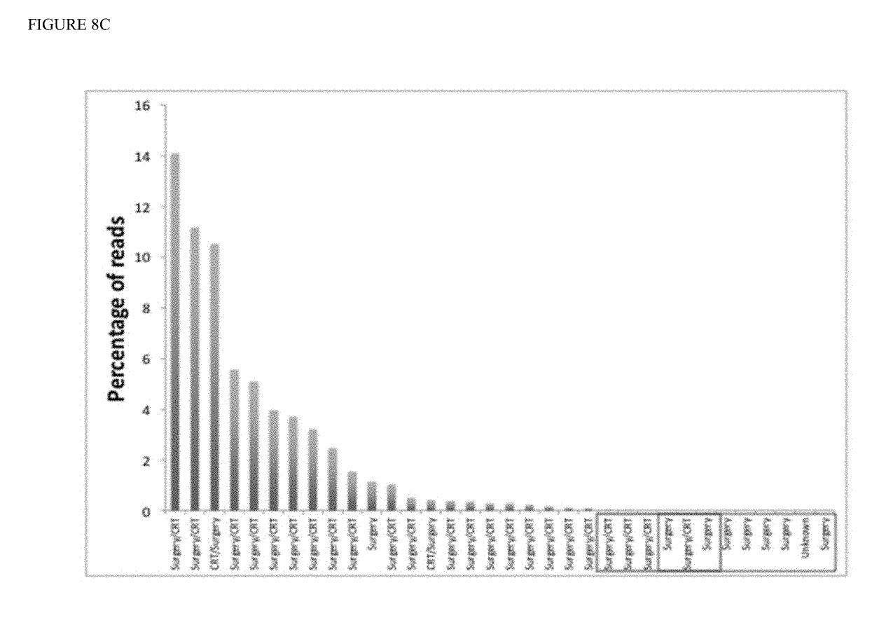

We similarly compared differentially enriched Fusobacterium species OTUs between control and HNSCC samples from Hopkins and HMP saliva, mid vagina and introitus (data not shown). This complex comparison revealed a high association of Fusobacterium nucleatum and Fusobacterium naviforme with specific HNSCC samples. Differences in abundance of their associative presence in HNSCC can be measured in comparison with their regular distribution across the normal oral microbiota of control patients, thus a noticeable increase is witnessed in HNSCC patient saliva samples. TNM-staging and HPV status color bars facilitate representation of their conditional association with the patient saliva samples. The relative enrichment of Fusobacterium nucleatum in HNSCC saliva when compared to controls from Hopkins and HMP (FIG. 8A) is only seen in patients with advanced disease (T3>) FIG. 8B) and only in patients treated with surgery and chemo-radio-therapy when compared with patients only treated with surgical removal of the tumor (FIG. 8C). When considering the coupled analysis of Resphera-assigned HNSCC-Hopkins and the Human Microbiome Project saliva and vaginal samples, a similar trend in the differential abundances of all Fusobacterium spp. is also witnessed, showing a comparable distribution and association with the HNSCC-Hopkins samples. Fusobacterium species such as F. canifelium, F. nucleatum and F. naviforme were differentially abundant across all samples.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Blaser, M. J., Understanding microbe-induced cancers. Cancer Prev Res (Phila), 2008. 1(1): p. 15-20.
2. Ohtani, N., Microbiome and cancer. Semin Immunopathol, 2015. 37(1): p. 65-72.
3. Maldonado-Contreras, A., et al., Structure of the human gastric bacterial community in relation to Helicobacter pylori status. ISMS J, 2011. 5(4): p. 574-9.
4. Lee, I. O., et al., Helicobacter pylori CagA phosphorylation status determines the gp130-activated SHP2/ERK and JAK/STAT signal transduction pathways in gastric epithelial cells. J Biol Chem, 2010. 285(21): p. 16042-50.
5. Brawner, K. M., C. D. Morrow, and P. D. Smith, Gastric microbiome and gastric cancer. Cancer J, 2014. 20(3): p. 211-6.
6. Vetizou, M., et al., Anticancer immunotherapy by CTLA-4 blockade relies on the gut microbiota. Science, 2015. 350(6264): p. 1079-84.
7. Kerr, A. R., The oral microbiome and cancer. J Dent Hyg, 2015. 89 Suppl 1: p. 20-3.
8. Michaud, D. S. and J. Izard, Microbiota, oral microbiome, and pancreatic cancer. Cancer J, 2014. 20(3): p. 203-6.
9. Narayanan, V., M. P. Peppelenbosch, and S. R. Konstantinov, Human fecal microbiome-based biomarkers for colorectal cancer. Cancer Prev Res (Phila), 2014. 7(11): p. 1108-11.
10. Weir, T. L., et al., Stool microbiome and metabolome differences between colorectal cancer patients and healthy adults. PLoS One, 2013. 8(8): p. e70803.
11. Geng, J., et al., Diversified pattern of the human colorectal cancer microbiome. Gut Pathog, 2013. 5(1): p. 2.
12. Zambirinis, C. P., et al., Pancreatic cancer, inflammation, and microbiome. Cancer J, 2014. 20(3): p. 195-202.
13. Seki, E., Microbiome-obesity-liver cancer interaction: senescence of hepatic stellate cells and bile acids play new roles. Gastroenterology, 2014. 146(3): p. 860-1.
14. Cook, M. B., et al., Serum pepsinogens and Helicobacter pylori in relation to the risk of esophageal squamous cell carcinoma in the alpha-tocopherol, beta-carotene cancer prevention study. Cancer Epidemiol Biomarkers Prev, 2010. 19(8): p. 1966-75.
15. Blaser, M. J., Helicobacter pylori and esophageal disease: wake-up call? Gastroenterology, 2010. 139(6): p. 1819-22.
16. Plottel, C. S. and M. J. Blaser, Microbiome and malignancy. Cell Host Microbe, 2011. 10(4): p. 324-35.
17. Wynendaele, E., et al., Crosstalk between the microbiome and cancer cells by quorum sensing peptides. Peptides, 2015. 64: p. 40-8.
18. Shalapour, S. and M. Karin, Immunity, inflammation, and cancer: an eternal fight between good and evil. J Clin Invest, 2015. 125(9): p. 3347-55.
19. Grivennikov, S. I., F. R. Greten, and M. Karin, Immunity, inflammation, and cancer. Cell, 2010. 140(6): p. 883-99.
20. Shalapour, S., et al., Immunosuppressive plasma cells impede T-cell-dependent immunogenic chemotherapy. Nature, 2015. 521(7550): p. 94-8.
21. Takeda, K. and S. Akira, Toll-like receptors in innate immunity. Int Immunol, 2005. 17(1): p. 1-14.
22. Abreu, M. T., Toll-like receptor signalling in the intestinal epithelium: how bacterial recognition shapes intestinal function. Nat Rev Immunol, 2010. 10(2): p. 131-44.
23. Cheesman, S. E., et al., Epithelial cell proliferation in the developing zebrafish intestine is regulated by the Wnt pathway and microbial signaling via Myd88. Proc Natl Acad Sci USA, 2011. 108 Suppl 1: p. 4570-7.
24. Brubaker, S. W., et al., Innate immune pattern recognition: a cell biological perspective. Annu Rev Immunol, 2015. 33: p. 257-90.
25. Vande Voorde, J., J. Balzarini, and S. Liekens, An emerging understanding of the Janus face of the human microbiome: enhancement versus impairment of cancer therapy. J Antimicrob Chemother, 2014. 69(10): p. 2878-80.
26. Plottel, C. S., From the guest editor: beyond symbiosis: a cancer-centric view of the microbiome. Cancer J, 2014. 20(3): p. 167-9.
27. Hultman, S. J., Emerging roles of the microbiome in cancer. Carcinogenesis, 2014. 35(2): p. 249-55.
28. Human Microbiome Project, C., A framework for human microbiome research. Nature, 2012.486(7402): p. 215-21.
29. Hamady, M. and R. Knight, Microbial community profiling for human microbiome projects: Tools, techniques, and challenges. Genome Res, 2009. 19(7): p. 1141-52.
30. Wang, L. and I. Ganly, The oral microbiome and oral cancer. Clin Lab Med, 2014. 34(4): p. 711-9.
31. Jumpstart Consortium Human Microbiome Project Data Generation Working, G., Evaluation of 16S rDNA-based community profiling for human microbiome research. PLoS One, 2012. 7(6): p. e39315.
32. Ahn, J., et al., Human gut microbiome and risk for colorectal cancer. J Natl Cancer Inst, 2013. 105(24): p. 1907-11.
33. Ahn, J., et al., Oral microbiome profiles: 16S rRNA pyrosequencing and microarray assay comparison. PLoS One, 2011. 6(7): p. e22788.
34. Ferris, R. L., Immunology and Immunotherapy of Head and Neck Cancer. J Clin Oncol, 2015.
35. Guo, T. and J. A. Califano, Molecular biology and immunology of head and neck cancer. Surg Oncol Clin N Am, 2015. 24(3): p. 397-407.
36. Reed, A. L., et al., High frequency of p16 (CDKN2/MTS-1/INK4A) inactivation in head and neck squamous cell carcinoma. Cancer Res, 1996. 56(16): p. 3630-3.
37. Koch, W. M., et al., Head and neck cancer in nonsmokers: a distinct clinical and molecular entity. Laryngoscope, 1999. 109(10): p. 1544-51.
38. Gillison, M. L., et al., Evidence for a causal association between human papillomavirus and a subset of head and neck cancers. J Natl Cancer Inst, 2000. 92(9): p. 709-20.
39. Tokumaru, Y., et al., Inverse correlation between cyclin A1 hypermethylation and p53 mutation in head and neck cancer identified by reversal of epigenetic silencing. Cancer Res, 2004. 64(17): p. 5982-7.
40. Ha, P. K. and J. A. Califano, Promoter methylation and inactivation of tumour-suppressor genes in oral squamous-cell carcinoma. Lancet Oncol, 2006. 7(1): p. 77-82.
41. Agrawal, N., et al., Exome sequencing of head and neck squamous cell carcinoma reveals inactivating mutations in NOTCH1. Science, 2011. 333(6046): p. 1154-7.
42. Poeta, M. L., et al., TP53 mutations and survival in squamous-cell carcinoma of the head and neck. N Engl J Med, 2007. 357(25): p. 2552-61.
43. Guerrero-Preston, R., et al., Key tumor suppressor genes inactivated by “greater promoter” methylation and somatic mutations in head and neck cancer. Epigenetics, 2014. 9(7): p. 1031-46.
44. Sun, W., et al., Activation of the NOTCH pathway in head and neck cancer. Cancer Res, 2014. 74(4): p. 1091-104.
45. Gaykalova, D. A., et al., NF-kappaB and stat3 transcription factor signatures differentiate HPV-positive and HPV-negative head and neck squamous cell carcinoma. Int J Cancer, 2015. 137(8): p. 1879-89.
46. Cancer Genome Atlas, N., Comprehensive genomic characterization of head and neck squamous cell carcinomas. Nature, 2015. 517(7536): p. 576-82.
47. Guerrero-Preston, R., et al., 16S rRNA amplicon sequencing identifies microbiota associated with oral cancer, Human Papilloma Virus infection and surgical treatment. Oncotarget, 2016.
48. Hogue, M. O., et al., Genome-wide genetic characterization of bladder cancer: a comparison of high-density single-nucleotide polymorphism arrays and PCR-based microsatellite analysis. Cancer Res, 2003. 63(9): p. 2216-22.
49. Kuczynski, J., et al., Using QIIME to analyze 16S rRNA gene sequences from microbial communities. Curr Protoc Bioinformatics, 2011. Chapter 10: p. Unit 10 7.
50. Navas-Molina, J. A., et al., Advancing our understanding of the human microbiome using QIIME. Methods Enzymol, 2013. 531: p. 371-444.
51. Shannon, P., et al., Cytoscape: a software environment for integrated models of biomolecular interaction networks. Genome Res, 2003. 13(11): p. 2498-504.
52. Ottesen, A., et al., Enrichment dynamics of Listeria monocytogenes and the associated microbiome from naturally contaminated ice cream linked to a listeriosis outbreak. 2016: Submitted.
53. Daquigan, N. Evaluating Food Enrichment Methods Using the Illumina MiSeq. in IAFP's 12th European Symposium on Food Safety. 2016. Iafp.
54. Pfefer, T. Evaluation of Enriched Microflora of Raw Milk Cheese Spiked with E. coli 0157: H7 and E. coli 0103 using Next-Generation Sequencing Technology. in IAFP 2016 Annual Meeting. 2016. Iafp.
55. White, J. R., J. Drewes, and C. L. Sears, High-resolution microbiome profiling and meta-analysis yields insight into microbial consortia associated with colorectal cancer. Cancer Research, 2016. 76(14 Supplement): p. 844-844.
56. Human Microbiome Project, C., Structure, function and diversity of the healthy human microbiome. Nature, 2012. 486(7402): p. 207-14.
57. Oksanen, J., et al., vegan: Community Ecology Package. R package version 1.17-2. CRAN.R-project.org/package=vegan, 2008: p. available in: vegan.r-forge.r-project.org
58. McMurdie, P. J. and S. Holmes, phyloseq: an R package for reproducible interactive analysis and graphics of microbiome census data. PLoS One, 2013. 8(4): p. e61217.
59. Segata, N., et al., Metagenomic biomarker discovery and explanation. Genome Biol, 2011. 12(6): p. R60.

1. A method for identifying the bacterial taxonomic profile in a subject suspected of having HNSCC, or having HNSCC, comprising:
a) obtaining a biological sample from the subject;
b) isolating the nucleic acid in the sample;
c) amplifying the 16S rRNA V3-V5 gene region of bacterial nucleic acid present in the sample of b) using specific primers and probes for the 16S rRNA V3-V5 region;
d) sequencing the amplified DNA of step c); and
e) identifying the bacterial species present in the sample using high-resolution profiling of the sequences of step d).

2. The method of claim 1, wherein the test sample contains oral squamous cells or nucleic acids from oral squamous cells.

3. The method of claim 2, wherein the identification of at least two bacterial species are detected.

4. The method of claim 2, wherein the identification of at least three bacterial species is detected.

5. The method of claim 2, wherein the identification of at least four bacterial species is detected.

6. A method for triaging a patient suspected of having as having Head and Neck Squamous Cell Cancer (HNSCC) into treatment comprising the steps of:
a) obtaining a biological sample from the subject;
b) isolating the nucleic acid in the sample;
c) amplifying the 16S rRNA V3-V5 gene region of bacterial nucleic acid present in the sample of b) using specific primers and probes for the 16S rRNA V3-V5 region;
d) sequencing the amplified DNA of step c);
e) identifying the bacterial species present in the sample using high-resolution profiling of the sequences of step d); and
f) triaging the subject suspected of having HNSCC into treatment for HNSCC when one or more bacterial species selected from the group consisting of Lactobacillus gasseri, Lactobacillus johnsonii, Haemophilus parainfluenza, Lactobacillus fermentum, L. rhamnosus and Fusobacterium periodonticum is present in the sample.

7. The method of claim 6, wherein the test sample contains oral squamous cells or nucleic acids from oral squamous cells.

8. The method of claim 7, wherein the identification of at least two bacterial species are detected.

9. The method of claim 7, wherein the identification of at least three bacterial species is detected.

10. The method of claim 7, wherein the identification of at least four bacterial species is detected.

11. A method of triaging a subject having Head and Neck Squamous Cell Cancer (HNSCC) into treatment for oropharyngeal squamous cell cancer (OPSCC) comprising the steps of:
a) obtaining a biological sample from the subject;
b) isolating the nucleic acid in the sample;
c) amplifying the 16S rRNA V3-V5 gene region of bacterial nucleic acid present in the sample of b) using specific primers and probes for the 16S rRNA V3-V5 region;
d) sequencing the amplified DNA of step c);
e) identifying the bacterial species present in the sample using high-resolution profiling of the sequences of step d);
f) triaging the subject into treatment for OPSCC when one or more bacterial species selected from the group consisting of Lactobacillus gasseri, and Lactobacillus johnsonii.

12. The method of claim 11, wherein the test sample contains oral squamous cells or nucleic acids from oral squamous cells.

13. The method of claim 12, wherein the identification of at least two bacterial species are detected.

14. The method of claim 12, wherein the identification of at least three bacterial species is detected.

15. The method of claim 12, wherein the identification of at least four bacterial species is detected.
